(19) United States
(12) Patent Application Publication
(10) Pub. No.: US 2023/0152325 A1
(43) Pub. Date: May 18, 2023

(54) Active Alpha-5-Beta-1 Integrin As A Biomarker For Enhancing Tumor Treatment Efficacy

(71) Applicant: Institute For Cancer Research d/b/a The Research Institute Of Fox Chase Cancer Center, Philadelphia, PA (US)

(72) Inventors: Edna Cukierman, Philadelphia, PA (US); Janusz Franco-Barraza, Philadelphia, PA (US); Neelima Shah, Philadelphia, PA (US)

(21) Appl. No.: 16/310,566

(22) PCT Filed: Jun. 26, 2017
(86) PCT No.: PCT/US17/39266

§ 371 Date: Dec. 17, 2018

Related U.S. Application Data
(60) Provisional application No. 62/354,854, filed on Jun. 27, 2016.

(51) Int. Cl. G01N 33/574

(52) U.S. Cl. CPC G01N 33/57492; G01N 2333/91205; G01N 2333/7055

(57) ABSTRACT

Methods for intervening in conditions of active desmoplasia comprise detecting increased levels of active alpha5-beta-1 integrin and the localization of this active integrin intracellularly away from three dimensional matrix adhesions within the stroma of affected tissues, as well as detecting the concomitant enhanced activity of focal adhesion kinase. Once active desmoplasia is detected, treatments may ensue, which induce a desmoplastic extracellular matrix to revert to a normal/innate phenotype, or which alter the standard of care to improve an outcome that would be less beneficial without the detection of a treatment-impeding desmoplasia condition. Liquid biopsies for detecting active desmoplasia are provided.

### STATEMENT OF GOVERNMENT SUPPORT

This invention was made, in part, with government support under Grant No. R01CA113451 awarded by U.S. National Institutes of Health. The government has certain rights in the invention.

### FIELD

The disclosure relates generally to the field of cancer and chronic fibrosis treatments. More particularly, the disclosure relates to detecting active alpha-5-beta-1 integrin as a biomarker of active desmoplasia and of chronic fibrosis, which prompts treatment to revert the active desmoplasia to a normal phenotype, or which prompts an alteration of a standard of care that would have reduced efficacy due to the presence of active desmoplasia.

### BACKGROUND

Various publications, including patents, published applications, accession numbers, technical articles and scholarly articles are cited throughout the specification. Each of these cited publications is incorporated by reference, in its entirety and for all purposes, in this document.

Desmoplastic stroma plays an important role in epithelial (e.g., pancreatic or renal) tumor development and progression. A vast portion of mesenchymal stroma or desmoplasia-related literature suggest a chronic wound healing-like pro-tumorigenic role for desmoplasia, while other reports propose a tumor protective and/or drug impenetrable role. Nonetheless, homeostatic normal/innate mesenchymal stroma is considered a natural tumor suppressive microenvironment. Evidence has emerged that desmoplastic ablation is detrimental to patients. Other evidence indicates that reprograming mesenchymal stroma back to its restrictive innate state seems to bear therapeutic promise, including reinstituting anti-tumoral immune activity. Thus, desmoplasia has potential implications for tumor therapy. There remains a need in the art to be able to manipulate desmoplastic stroma, toward improving the understanding of the underlying biology behind stromal activation.

It was previously shown that extracellular matrices (ECMs) produced by fibroblastic naïve and activated cells (e.g., tumor- or cancer-associated fibroblasts; known as TAFs or CAFs), respectively present phenotypes that are reminiscent of in vivo quiescent tumor restrictive and activated/desmoplastic stroma. Similarly, it was previously demonstrated that desmoplastic ECM (D-ECM) can serve as pathophysiological relevant substrates capable of activating naïve/innate fibroblasts. There further remains a need to harness the role that desmoplasia plays in cancer development and progression, toward improving patient outcomes.

### SUMMARY

The present disclosure provides methods comprising isolating desmoplastic stroma from one or more of the pancreas, kidney, lung, or other epithelial tissue that may bear desmoplastic reaction, of a human subject, detecting increased levels of active alpha-5-beta-1 integrin localized intracellularly away from three dimensional matrix adhesions and/or increased re-localization of this active integrin to 3D-adhesions, and detecting increased focal adhesion kinase activity in the stroma, and then treating the subject with a therapeutic regimen that inhibits one or more of fibroblast activation, the biologic activity of transforming growth factor (TGF) beta, or the biologic activity of alpha-5-beta-1 integrin in one or more of the pancreatic stroma, kidney stroma, lung stroma, or stroma of other tissue, or regimen that blocks chronic fibrosis or inflammation, thereby inducing the desmoplastic stroma to express a normal/innate phenotype. In some embodiments, the treatment comprises administering to the subject vitamin D or a vitamin D analog, or impeding the biological activity of transforming growth factor (TGF) beta, or impeding the biologic activity of alpha-v-beta-5 integrin, or other anti-fibrotic treatments, using biochemical or small molecules inhibitors in an amount effective to induce the desmoplastic stroma to switch to and/or express a normal/innate phenotype. In some embodiments, increased levels of active alpha-5-beta-1 integrin is detected by contacting the isolated desmoplastic stroma with an antibody that specifically binds to an active conformation of alpha-5-beta-1 integrin. In some embodiments, the antibody that specifically binds to an active conformation of alpha-5-beta-1 integrin comprises SNAKA51.

In some embodiments, isolating desmoplastic stroma comprises taking a biopsy of one or more of the pancreas, kidney, or lung. The biopsy may comprise a core needle biopsy or a surgical biopsy. The pancreas, kidney, or lung being assessed in the subject may be cancerous.

The present disclosure also provides methods comprising isolating micro vesicles and/or exosomes from the blood and/or ascites fluid (or any other suitable biologic fluid such as saliva, gingival crevicular fluid, urine, sweat, tears, spinal fluid, etc.) of a human subject, detecting active alpha-5-beta-1 integrin (levels, localization and/or co-expression with active FAK and other proteins indicative of desmoplasia and/or chronic fibrosis) in the micro vesicle and/or exosome, and then treating the subject with a therapeutic regimen that inhibits one or more of fibroblast activation, the biologic activity of transforming growth factor (TGF) beta, or the biologic activity of alpha-5-beta-1 integrin in desmoplastic stroma, or regimen that blocks chronic fibrosis or inflammation, thereby inducing the desmoplastic stroma to express a normal phenotype. In some embodiments, the treatment comprises administering to the subject vitamin D or a vitamin D analog in an amount effective to induce the desmoplastic stroma to express a normal/innate phenotype. The treatment may comprise administering to the subject vitamin D or a vitamin D analog, or impeding the biological activity of TGF beta, or impeding the biologic activity of alpha-v-beta-5 integrin, or other anti-fibrotic treatments, using biochemical or small molecules inhibitors in an amount effective to induce the desmoplastic stroma to switch to a normal/innate phenotype. In some embodiments, the method comprise solubilizing the micro vesicle and/or solubilizing the exosome or detecting active alpha-5-beta-1 integrin at the membrane of the extracellular vesicle (e.g., micro vesicle and/or the exosome) with an antibody that specifically binds to an active conformation of alpha-5-beta-1 integrin. In some embodiments, the antibody that specifically binds to an active conformation of alpha-5-beta-1 integrin comprises SNAKA51.

Such liquid biopsies, which generally are less invasive than solid tissue biopsies, may be used to detect the presence of active desmoplasia in one or more of the pancreas, kidney, or lung of the subject. The pancreas, kidney, or lung being assessed in this way may be cancerous.

The present disclosure also provides methods comprising isolating desmoplastic stroma from one or more of a cancerous pancreas, cancerous kidney, or cancerous lung, or other cancerous epithelial tissue of a human subject, detecting increased levels of active alpha-5-beta-1 integrin localized intracellularly away from three dimensional matrix adhesions and/or increased re-localization of this active integrin to 3D-adhesions, and detecting increased focal adhesion kinase activity in the stroma, and administering to the subject an amount of interferon gamma effective to prime T lymphocytes. In some embodiments, the methods comprise re-isolating desmoplastic stroma from one or more of the cancerous pancreas, cancerous kidney, or cancerous lung or other cancerous epithelial tissue from the subject after a period of time following the administering of interferon gamma, detecting decreased levels of active alpha-5-beta-1 integrin localized intracellularly away from three dimensional matrix adhesions and detecting decreased focal adhesion kinase activity in the stroma, and administering to the subject an effective amount of an antibody that specifically binds to PD-1 or to PD-L1, thereby treating the cancerous pancreas, cancerous kidney, or cancerous lung, or other cancerous epithelial tissue. In some embodiments, the methods further comprise treating the subject with a therapeutic regimen that inhibits one or more of fibroblast activation, the biologic activity of transforming growth factor (TGF) beta, or the biologic activity of alpha-5-beta-1 integrin in pancreatic stroma, kidney stroma, or lung stroma, or regimen that blocks chronic fibrosis or inflammation, thereby inducing the desmoplastic stroma to express a normal/innate phenotype. In some embodiments, such treatment comprises administering to the subject vitamin D or a vitamin D analog, or impeding the biological activity of transforming growth factor (TGF) beta, or impeding the biologic activity of alpha-v-beta-5 integrin, or other anti-fibrotic treatments, using biochemical or small molecules inhibitors in an amount effective to induce the desmoplastic stroma to switch to a normal/innate phenotype. In some embodiments, detection of increased or decreased levels of active alpha-5-beta-1 integrin comprises contacting the isolated desmoplastic stroma with an antibody that specifically binds to an active conformation of alpha-5-beta-1 integrin, such as SNAKA51.

Isolating and re-isolating desmoplastic stroma may comprise taking a biopsy of one or more of the cancerous pancreas, kidney, or lung or other cancerous epithelial tissue. The biopsy may comprise a core needle biopsy or a surgical biopsy.

The present disclosure also provides methods comprising isolating micro vesicles and/or exosomes from the blood and/or ascites fluid (or any other suitable biologic fluid such as saliva, gingival crevicular fluid, urine, sweat, tears, spinal fluid, etc.) of a human subject, detecting increased general levels of active alpha-5-beta-1 integrin in the stromal or fibrous micro vesicle or exosome, and administering to the subject an amount of interferon gamma effective to prime T lymphocytes, or other agent suitable for priming T lymphocytes. In some embodiments, the methods comprise isolating a second micro vesicle or exosome from the blood or ascites fluid (or any other suitable biologic fluid such as saliva, gingival crevicular fluid, urine, sweat, tears, spinal fluid, etc.) of a human subject after a period of time following the administering of interferon gamma (or any other agent suitable for priming T lymphocytes or other immune cells), detecting decreased vesicular levels of active alpha-5-beta-1 integrin in the micro vesicle or exosome, and administering to the subject an effective amount of an antibody that specifically binds to PD-1 or to PD-L1 (or similar immune checkpoint inhibitors), thereby treating the cancerous pancreas, cancerous kidney, or cancerous lung. In some embodiments, the methods further comprise treating the subject with a therapeutic regimen that inhibits one or more of fibroblast activation, the biologic activity of transforming growth factor (TGF) beta, or the biologic activity of alpha-5-beta-1 integrin in pancreatic stroma, kidney stroma, or lung stroma, or regimen that blocks chronic fibrosis or inflammation, thereby inducing the desmoplastic stroma to express a normal phenotype. In some embodiments, such treatment comprises administering to the subject vitamin D or a vitamin D analog, or impeding the biological activity of TGF beta, or impeding the biologic activity of alpha-v-beta-5 integrin, or other anti-fibrotic treatments, using biochemical or small molecules inhibitors in an amount effective to induce the desmoplastic stroma to switch to a normal/innate phenotype. In some embodiments, detection of increased or decreased levels of active alpha-5-beta-1 integrin comprises contacting the isolated desmoplastic stroma with an antibody that specifically binds to an active conformation of alpha-5-beta-1 integrin, such as SNAKA51. In some embodiments, the methods comprise immuno-isolating (e.g., to select for SNAKA51-positive vesicles) and/or solubilizing the micro vesicle and/or solubilizing the exosome.

### BRIEF DESCRIPTION OF THE DRAWINGS

FIGS. 1A through 1E show desmoplastic (D)-ECM remodeling by TGFβ-blockage bestows normal (N)-ECM-like phenotypes.

FIGS. 2A through 2D show that alpha-v-beta-5 (αvβ5) integrin regulates alpha-5-beta-5 (α5β1) integrin activity, thus maintaining D-ECM-induced myofibroblastic/desmoplastic phenotype in a TGFβ-independent manner.

FIGS. 3A through 3H show FAK independent α5β1 integrin activity negatively regulates D-ECM-induced desmoplastic activation.

FIGS. 4A through 4M show molecular alterations in three dimensional matrix adhesions (3D-adhesions) phenotype, as well as αvβ5 integrin-regulated redistribution of active α5β1 integrin to intracellular locations, which constitute trademarks of D-ECM induced responses.

FIGS. 5A through SI show the effect of genetically-nullified integrin gene (β KO) in fibroblasts reacting to D-ECMs and in D-ECM production (showing only PDAC associated fibroblasts), and loss of β5 integrin expression in CAFs impairs D-ECM alignment, while expression of α5 integrin is imperative for effective ECM fibrillogenesis.

FIGS. 6A through 6I show in vivo validations of in vitro uncovered phenotypes suggesting that levels and distributions of active α5β1 integrin and pFAK can effectively predict human PDAC and RCC recurrences.

FIGS. 7A through 7F show characterization of human fibroblastic cells isolated from PDAC and RCC surgical samples.

FIGS. 8A through 8D show αvβ5 integrin inhibition or α5β1 integrin stabilization during D-ECM production failed to alter the resulting ECMs.

FIGS. 9A through 9C show SMIA analysis of in vitro PDAC (top) and RCC (bottom), normal and desmoplastic fibroblasts, graphs summarizing normalized mean total intensity levels of active α5β1 integrin (left bullets), pFAK (center bullets) and pSMAD (right bullets), and analysis of samples displaying mean intensity of active α5β1 integrin distributed away from 3D-adhesions (left bullets), pFAK at 3D-adhesions (center bullets), or pSMAD at nuclear compartments (right bullets).

FIGS. 10A through 10C depict images of isolated cell-secreted microvesicles/exosomes containing active α5β1 integrin at the vesicle membrane.

FIG. 11 shows active α5β1 integrin is intracellularly localized at multi-vesicular bodies of activated CAFs (C2), marked by CD81, and that loss of CD81 prompts the relocaltion of the integrin at the plasma membrane (C1; representing normalized fibroblasts akin to αvβ5 inhibition).

### DESCRIPTION OF EMBODIMENTS

Various terms relating to embodiments of the present disclosure are used throughout the specification and claims. Such terms are to be given their ordinary meaning in the art, unless otherwise indicated. Other specifically defined terms are to be construed in a manner consistent with the definition provided in this document.

As used throughout, the singular forms “a,” “an,” and “the” include plural referents unless expressly stated otherwise.

A material has been “isolated” if it has been removed from its natural environment and/or altered by the hand of a human being.

The terms “subject” and “patient” are used interchangeably. A subject may be any animal, such as a mammal. A mammalian subject may be a farm animal (e.g., sheep, horse, cow, pig), a companion animal (e.g., cat, dog), a rodent or laboratory animal (e.g., mouse, rat, rabbit), or a non-human primate (e.g., old world monkey, new world monkey). In some embodiments, the mammal is a human.

Using pancreatic ductal adenocarcinoma (PDAC) and renal cell carcinoma (RCC) as well as a murine skin stromal models, the experiments described herein assessed the potential dynamic manipulation of both D-ECMs and their ability to induce fibroblastic activation. It was observed that the levels and localization of the active conformation of alpha5-beta-1 integrin together with levels of FAK activity (phosphor FAK) may serve as a biomarker indicative of active desmoplasia, which is patient-detrimental. Similarly high active integrin localized at 3D-adhesion structures represents the “protective” phenotype which is predictive of disease recurrence in pancreatic cancer patients following surgery. This biomarker combinatorial approach may be used to detect the development of early stage cancers associated with fibrosis predisposition, fibrosis per se, or to assess patient risk, as well as to predict the efficacy of chemotherapy or to alter a treatment course to enhance efficacy that would have been impaired given the presence of active desmoplasia, as well as to predict tumor recurrence. Identification of active desmoplasia or fibrosis may be further useful, for example, in distinguishing between benign stroma (high active alpha5-beta-1 integrin localized at (as opposed to away from) 3D-matrix adhesions) and detrimental desmoplasia. It was further observed that the detection of active desmoplasia (via the level and localization of the active conformation of alpha5-beta-1 integrin) coupled with increased focal adhesion kinase (FAK) activity can be used to stage desmoplasia. Accordingly, the present disclosure provides methods for reverting active, detrimental desmoplasia to a normal/innate phenotype, as well as methods for treating tumors associated with fibrosis or fibrosis per se. Any of the methods may be carried out in vivo, in vitro, or in situ.

The present disclosure provides methods for reverting active, detrimental desmoplasia to a normal phenotype comprising detecting increased levels of active alpha-5-beta-1 integrin localized intracellularly away from three dimensional matrix adhesions in the stroma isolated from a subject, and/or detecting increased re-localization of this active integrin to 3D-adhesions, and detecting increased focal adhesion kinase activity in the stroma isolated from a subject, thereby determining whether the isolated stroma includes active desmoplasia. If elevated levels of active alpha-5-beta-1 integrin (as a proxy for active desmoplasia) is detected, then the subject is treated with a therapeutic regimen that inhibits one or more of fibroblast activation, the biologic activity of transforming growth factor (TGF) beta, or the biologic activity of alpha-v-beta-5 integrin in the stroma. Such treatments induce the desmoplastic stroma to express a normal/innate phenotype. Such treatments may include administration of vitamin D or vitamin D analogs to the subject, in an amount effective to induce the desmoplastic stroma to express a normal/innate phenotype, or impeding the biological activity of transforming growth factor (TGF) beta, or impeding the biologic activity of alpha-v-beta-5 integrin, or other anti-fibrotic treatments, using biochemical or small molecules inhibitors in an amount effective to induce the desmoplastic stroma to switch to and/or express a normal/innate phenotype.

The stroma may be isolated from any suitable tissue in the subject. The tissue may comprise pancreatic tissue (e.g., pancreatic stroma), kidney tissue (e.g., kidney stroma), or lung tissue (e.g., lung stroma). The tissue may comprise any tissue, including epithelial tissue, from any organ, having stroma. The tissue may be fibrotic, cancerous, or may be pre-cancerous, or may be suspected of being cancerous or pre-cancerous. The assessment of the stroma need not be related to cancer, for example, the stroma may be isolated for determining a fibrosis condition, including pulmonary or lung fibrosis, renal/kidney fibrosis, or pancreatic fibrosis.

The detection of active alpha-5-beta-1 integrin may comprise contacting the isolated stroma with an antibody that specifically binds to an active conformation of alpha-5-beta-1 integrin. Such an antibody may comprise SNAKA51, or any antigen-binding fragment thereof. The SNAKA51 antibody or antigen-binding fragment thereof can discriminate between the active conformation of alpha-5-beta-1 integrin, to which the antibody specifically binds, and other conformations to which the antibody does not bind. SNAKA51 thus can distinguish between benign stroma and detrimental active desmoplasia and/or fibrosis. Before screening with the antibody, the isolated stroma may be digested with appropriate enzymes to aid in retrieving the antigen and maintain the integrin in its conformation.

Stroma may be isolated from the tissue according to any suitable isolation technique. A biopsy may be used in some embodiments. For example, a surgical biopsy may be used, or a core needle biopsy may be used.

In addition to or in the alternative to solid tissue sampling, the assessments of active alpha5-beta-1 integrin may be carried out in liquid samples, including liquid biopsies. Thus, in some embodiments, liquid biopsies are obtained from the subject.

In some embodiments of a liquid biopsy, the blood or ascites fluid, or any other suitable biologic fluid such as saliva, gingival crevicular fluid, urine, sweat, tears, spinal fluid, etc. of a subject may be isolated and screened. From such isolated biologic fluids, stromal micro vesicles and/or exosomes may be isolated. In either case, the biopsy methods comprise detecting active alpha-5-beta-1 integrin in a micro vesicle or exosome from the blood or ascites fluid (or other suitable biologic fluid), thereby determining whether stroma in the subject includes active or protective desmoplasia. In some embodiments, if active alpha-5-beta-1 integrin is detected, particularly concomitantly with the detection of high FAK activity levels (as a proxy for active desmoplasia), then the subject is treated with a therapeutic regimen that inhibits one or more of fibroblast activation, the biologic activity of transforming growth factor (TGF) beta, or the biologic activity of alpha-v-beta-5 integrin in the stroma. Such treatments induce the desmoplastic stroma to express a normal/innate phenotype. Such treatments may include administration of vitamin D or vitamin D analogs to the subject, in an amount effective to induce the desmoplastic stroma to express a normal/innate phenotype, or impeding the biological activity of transforming growth factor (TGF) beta, or impeding the biologic activity of alpha-v-beta-5 integrin, or other anti-fibrotic treatments, using biochemical or small molecules inhibitors in an amount effective to induce the desmoplastic stroma to switch to and/or express a normal/innate phenotype.

In some embodiments, the methods further comprise solubilizing the micro vesicle. In some embodiments, the methods further comprise solubilizing the exosome.

From the liquid biopsies, the detection of active alpha-5-beta-1 integrin may comprise contacting the microsome or exosome, or contents thereof, with an antibody that specifically binds to an active conformation of alpha-5-beta-1 integrin. Such an antibody may comprise SNAKA51, or any antigen-binding fragment thereof. Before screening with the antibody, the liquid biopsy may be treated with appropriate detergents (e.g., Triton®, SDS, NP 40, or other suitable detergent).

The liquid biopsies assess exosomal or vesicles shed from the stroma. In some embodiments, when liquid biopsies are screened, the methods may or may not include FAK activity assessments. The microvesicles/exosomes and/or any other type of extracellular vesicles may be shed from the stroma of a cancerous or pre-cancerous tissue. The tissue may be any tissue from which stroma may shed exosomes or microvesicles, and may include pancreatic tissue, kidney tissue, or lung tissue. Thus, the treatments may be directed to desmoplastic stroma as pancreatic desmoplastic stroma, kidney desmoplastic stroma, or lung desmoplastic stroma, or any other desmoplastic stroma.

It is believed that high endogenous (e.g., intracellular) levels of active alpha5-beta-1 integrin represent active detrimental desmoplasia or fibrosis. Thus, the detection of active desmoplasia may also be used to guide treatments of other conditions, including cancer or a fibrosis condition. The levels of active alpha-5-beta-1 integrin may be monitored before, during, and after treatment, for example, to assist in guiding a therapeutic regimen (e.g., chemotherapy) or to assess the efficacy of treatments for reverting the active desmoplasia to a normal/innate phenotype. In such cases, the chemotherapy regimen may be adjusted to take into account high levels of active desmoplasia, or to take into account diminishing levels of active desmoplasia, for example, where treatments successfully revert the desmoplasia to a normal/innate phenotype. Adjustments include, for example, one or more of adjustments to dosing, administration scheduling, starting time, drug types, and other relevant considerations attendant to therapy.

Immune priming may be a part of a treatment regimen, and may affect desmoplasia and the stroma, thereby having implications for adjustments to the treatment regimen, or may indicate that the stroma has reverted from an immunosuppressive state to a state of anti-tumor immune competency. Without intending to be limited to any particular theory or mechanism of action, it is believed that immune priming may induce stroma changes, including a reduction in the level of active desmoplasia or an alteration on the stroma microstructure sufficient to diminish the immune privileged (or immunosuppressive) nature of activated stroma. It is believed that immune priming-induced stroma changes are reflected in decreasing levels of active alpha-5-beta-1 integrin and/or decreasing levels of FAK activity in the stroma.

Immune priming includes the pre-activation of immune cells that participate in cell-mediated immunity, including T cells and peripheral blood mononuclear cells (PBMC) such as macrophages and natural killer (NK) cells. Cell-mediated immunity constitutes an important defense against cancer, though many cancers exhibit countermeasures that thwart the immune system's efforts. Immune priming treatments generally include administration of an activator of the immune cells of interest to a subject in need thereof. Immune cytokines are may be used as the priming activator, including interferons.

In certain cancers, the PD-1/PD-L1/L2 pathway plays a role in the tumors αvoiding attack by the immune system. PD-1 inhibitors, a relatively new category of cancer therapeutics, enhance the immune system attack by inhibiting the tumor's capacity to escape immune destruction. PD-1 inhibiting antibodies such as nivolumab and pembrolizumab bind to PD-1 and inhibit the PD-1/PD-L1 pathway. Gamma interferon increases PD-L1 levels.

The immune privileged nature of active stroma (e.g., stroma with high levels of active integrin and active FAK) is believed to inhibit tumor-killing immune cells from accessing the tumor. In such cases, even PD-1/PD-L1/L2-ready cells (e.g., cells treated with inhibitors such as nivolumab and pembrolizumab) would still be impeded from tumor destruction by the stroma, particularly an active stroma. Accordingly, it is believed that immune priming, such as the priming achieved by gamma interferon treatment, can induce stromal changes sufficient to weaken the stroma impediment to immune system-mediated tumor destruction. In this way, PD-1/PD-L1/L2-ready cells can gain access to the tumor. But if not, then treatments such as αvβ5 integrin inhibition or other anti-fibrotic treatments (TGF-beta-like or other anti-inflammatory treatments) may assist.

As time is of the essence in any cancer treatment regimen, it is helpful to know when the tumors are most vulnerable or, on alternately, when the tumor's innate defenses such as an activated stroma will impede the regimen. Thus, detection of active stroma (e.g., active desmoplasia) will further the goal of identifying treatment impediments and/or of knowing when the tumor's defenses are down. In this respect, the active desmoplasia-detecting modalities of this disclosure are useful to furthering these goals. For example, the detecting modalities may be used to determine when the stroma is most likely to impede an immunotherapy treatment regimen (e.g., because the stroma is in a stage of active desmoplasia and is immune privileged), and may further be used to determine when the stroma or active desmoplasia has been diminished sufficiently to garner immunotherapy success.

The present disclosure also provides methods comprising detecting increased levels of active alpha-5-beta-1 integrin localized intracellularly away from three dimensional matrix adhesions in the stroma isolated from a subject having cancer, and detecting increased focal adhesion kinase activity in the stroma, thereby determining whether the isolated stroma includes active desmoplasia. If elevated levels of active alpha-5-beta-1 integrin (as a proxy for active desmoplasia) is detected, then the subject is treated with a therapeutic regimen that inhibits one or more of fibroblast activation, the biologic activity of transforming growth factor (TGF) beta, or the biologic activity of alpha-v-beta-5 integrin in the stroma. For example, the subject may be treated with anti-fibrotic and anti-inflammation treatment regimens. The subject may be treated with an amount of interferon, such as gamma interferon, effective to both prime immune cells such as T lymphocytes and to revert the desmoplastic stroma to express a normal phenotype. In some embodiments, after a period of time sufficient to at least prime the immune cells, stroma is again isolated from the subject, followed by detecting decreased levels of active alpha-5-beta-1 integrin localized intracellularly away from three dimensional matrix adhesions in the stroma and/or increased re-localization of this active integrin to 3-D adhesions, and detecting decreased focal adhesion kinase activity in the stroma, thereby determining whether the isolated stroma includes diminished active desmoplasia (e.g., relative to the increased levels of active desmoplasia before interferon treatment). If the levels of active desmoplasia are decreasing in the subject or benign, protective levels are increasing, the method comprises administering to the subject an effective amount of an antibody that specifically binds to PD-1 or to PD-L1, including nivolumab and/or pembrolizumab. Administering such an antibody thereby treats the cancer in the subject. The cancer may be any cancer capable of being treated with antibodies to PD-1 or PD-L1. The cancer may be kidney cancer, pancreatic cancer, or lung cancer.

The stroma may be isolated from cancerous tissue in the subject, for example, via a tissue biopsy. The tissue may comprise pancreatic tissue (e.g., pancreatic stroma), kidney tissue (e.g., kidney stroma), or lung tissue (e.g., lung stroma). The detection of increased or decreased active alpha-5-beta-1 integrin may comprise contacting the isolated stroma with an antibody that specifically binds to an active conformation of alpha-5-beta-1 integrin. Such an antibody may comprise SNAKA51, or any antigen-binding fragment thereof.

The present disclosure also provides methods comprising detecting increased levels active alpha-5-beta-1 integrin in the micro vesicle or exosome isolated from the blood or ascites fluid, or any other suitable biologic fluid such as saliva, gingival crevicular fluid, urine, sweat, tears, spinal fluid, etc., of a human subject having cancer, thereby determining whether the micro vesicle or exosome contains active desmoplasia. If elevated levels of active alpha-5-beta-1 integrin (as a proxy for active desmoplasia) is detected, then the subject is treated with a therapeutic regimen that inhibits one or more of fibroblast activation, the biologic activity of transforming growth factor (TGF) beta, or the biologic activity of alpha-v-beta-integrin in the stroma. The subject may be treated with an amount of interferon, such as gamma interferon, effective to both prime immune cells such as T lymphocytes and to revert the desmoplastic stroma to express a normal phenotype. In some embodiments, after a period of time sufficient to at least prime the immune cells, micro vesicles or exosomes are again isolated from the blood or ascites fluid (or any other suitable biologic fluid such as saliva, gingival crevicular fluid, urine, sweat, tears, spinal fluid, etc.) from the subject, followed by detecting increased levels active alpha-5-beta-1 integrin in the micro vesicle or exosome, thereby determining whether the micro vesicle or exosome contains diminished active desmoplasia (e.g., relative to the increased levels of active desmoplasia before interferon treatment). If the levels of active desmoplasia are decreasing or if benign protective levels are increasing in the subject, the method comprises administering to the subject an effective amount of an antibody that specifically binds to PD-1 or to PD-L1, including nivolumab and/or pembrolizumab (or any other similar treatment that may act similarly upon other cells like NK and macrophages). Administering such an antibody thereby treats the cancer in the subject. The cancer may be any cancer capable of being treated with antibodies to PD-1 or PD-L1. The cancer may be kidney cancer, pancreatic cancer, or lung cancer.

In some embodiments, the method comprises alpha-5-beta-1 detection following solubilizing the micro vesicle and/or the exosome. The detection of increased or decreased active alpha-5-beta-1 integrin may comprise contacting the contents of the micro vesicle and/or the exosome with an antibody that specifically binds to an active conformation of alpha-5-beta-1 integrin. Such an antibody may comprise SNAKA51, or any antigen-binding fragment thereof. The methods may also comprise detecting active FAK.

The following representative embodiments are presented:

Embodiment 1. A method, comprising isolating desmoplastic stroma from the pancreas of a human subject, detecting increased levels of active alpha-5-beta-1 integrin localized intracellularly away from three dimensional matrix adhesions, and detecting increased focal adhesion kinase activity in the stroma, and then treating the subject with a therapeutic regimen that inhibits one or more of fibroblast activation, the biologic activity of transforming growth factor (TGF) beta, or the biologic activity of alpha-v-beta-integrin in pancreatic stroma, or that blocks chronic fibrosis or inflammation, thereby inducing the desmoplastic stroma to express a normal phenotype.

Embodiment 2. The method according to embodiment 1, wherein the step of detecting increased levels of active alpha-5-beta-1 integrin comprises contacting the isolated desmoplastic stroma with an antibody that specifically binds to an active conformation of alpha-5-beta-1 integrin.

Embodiment 3. The method according to embodiment 2, wherein the antibody that specifically binds to an active conformation of alpha-5-beta-1 integrin is SNAKA51.

Embodiment 4. The method according to any one of embodiments 1 to 3, wherein isolating desmoplastic stroma from the pancreas of the subject comprises taking a biopsy of the pancreas.

Embodiment 5. The method according to embodiment 4, wherein the biopsy is a core needle biopsy.

Embodiment 6. The method according to embodiment 4, wherein the biopsy is a surgical biopsy.

Embodiment 7. The method according to any one of embodiments 1 to 6, wherein the pancreas is cancerous.

Embodiment 8. A method, comprising isolating desmoplastic stroma from the kidney of a human subject, detecting increased levels of active alpha-5-beta-1 integrin localized intracellularly away from three dimensional matrix adhesions, and detecting increased focal adhesion kinase activity in the stroma, and then treating the subject with a therapeutic regimen that inhibits one or more of fibroblast activation, the biologic activity of transforming growth factor (TGF) beta, or the biologic activity of alpha-v-beta-integrin in kidney stroma, thereby inducing the desmoplastic stroma, or that blocks chronic fibrosis or inflammation, to express a normal phenotype.

Embodiment 9. The method according to embodiment 8, wherein the step of detecting increased levels of active alpha-5-beta-1 integrin comprises contacting the isolated desmoplastic stroma with an antibody that specifically binds to an active conformation of alpha-5-beta-1 integrin.

Embodiment 10. The method according to embodiment 9, wherein the antibody that specifically binds to an active conformation of alpha-5-beta-1 integrin is SNAKA51.

Embodiment 11. The method according to any one of embodiments 8 to 10, wherein isolating desmoplastic stroma from the kidney of the subject comprises taking a biopsy of the kidney.

Embodiment 12. The method according to embodiment 11, wherein the biopsy is a core needle biopsy.

Embodiment 13. The method according to embodiment 11, wherein the biopsy is a surgical biopsy.

Embodiment 14. The method according to any one of embodiments 8 to 13, wherein the kidney is cancerous.

Embodiment 15. A method, comprising isolating a stromal micro vesicle or exosome from the blood or ascites fluid of a human subject, detecting active alpha-5-beta-1 integrin in the micro vesicle or exosome, and then treating the subject with a therapeutic regimen that inhibits one or more of fibroblast activation, the biologic activity of transforming growth factor (TGF) beta, or the biologic activity of alpha-v-beta-integrin in desmoplastic stroma, or that blocks chronic fibrosis or inflammation, thereby inducing the desmoplastic stroma to express a normal phenotype.

Embodiment 16. The method according to embodiment 15, wherein the method comprises isolating a micro vesicle from the blood of the human subject, detecting active alpha-5-beta-1 integrin in the micro vesicle, and then treating the subject with a therapeutic regimen that inhibits one or more of fibroblast activation, the biologic activity of transforming growth factor (TGF) beta, or the biologic activity of alpha-v-beta-integrin in desmoplastic stroma, thereby inducing the desmoplastic stroma to express a normal phenotype.

Embodiment 17. The method according to embodiment 15, wherein the method comprises isolating a micro vesicle from the ascites fluid of the human subject, detecting active alpha-5-beta-1 integrin in the micro vesicle, and then treating the subject with a therapeutic regimen that inhibits one or more of fibroblast activation, the biologic activity of transforming growth factor (TGF) beta, or the biologic activity of alpha-v-beta-integrin in desmoplastic stroma, thereby inducing the desmoplastic stroma to express a normal phenotype.

Embodiment 18. The method according to embodiment 15, wherein the method comprises isolating an exosome from the blood of the human subject, detecting active alpha-5-beta-1 integrin in the exosome, and then treating the subject with a therapeutic regimen that inhibits one or more of fibroblast activation, the biologic activity of transforming growth factor (TGF) beta, or the biologic activity of alpha-v-beta-integrin in desmoplastic stroma, thereby inducing the desmoplastic stroma to express a normal phenotype.

Embodiment 19. The method according to embodiment 15, wherein the method comprises isolating an exosome from the ascites fluid of the human subject, detecting active alpha-5-beta-1 integrin in the exosome, and then treating the subject with a therapeutic regimen that inhibits one or more of fibroblast activation, the biologic activity of transforming growth factor (TGF) beta, or the biologic activity of alpha-v-beta-integrin in desmoplastic stroma, thereby inducing the desmoplastic stroma to express a normal phenotype.

Embodiment 20. The method according to any one of embodiments 15 to 17, further comprising solubilizing the micro vesicle.

Embodiment 21. The method according to any one of embodiments 15, 18, or 19, further comprising solubilizing the exosome.

Embodiment 22. The method according to any one of embodiments 15 to 21, wherein the step of detecting active alpha-5-beta-1 integrin comprises contacting the internal contents of the micro vesicle or the exosome with an antibody that specifically binds to an active conformation of alpha-5-beta-1 integrin.

Embodiment 23. The method according to embodiment 22, wherein the antibody that specifically binds to an active conformation of alpha-5-beta-1 integrin is SNAKA51.

Embodiment 24. The method according to any one or embodiments 15 to 23, wherein the desmoplastic stroma is desmoplastic stroma from the pancreas.

Embodiment 25. The method according to any one of embodiments 15 to 24, wherein the subject has pancreatic cancer.

Embodiment 26. The method according to any one or embodiments 15 to 23, wherein the desmoplastic stroma is desmoplastic stroma from the kidney.

Embodiment 27. The method according to any one of embodiments 15 to 23 or 26, wherein the subject has kidney cancer.

Embodiment 28. A method, comprising isolating desmoplastic stroma from a cancerous pancreas of a human subject, detecting increased levels of active alpha-5-beta-1 integrin localized away from three dimensional matrix adhesions, detecting increased focal adhesion kinase activity in the stroma, and administering to the subject an amount of interferon gamma effective to prime T lymphocytes; after a period of time following the administering of interferon gamma, isolating desmoplastic stroma from the pancreas, detecting decreased levels of active alpha-5-beta-1 integrin localized away from three dimensional matrix adhesions, and detecting decreased focal adhesion kinase activity in the stroma, and administering to the subject an effective amount of an antibody that specifically binds to PD-1 or to PD-L1, thereby treating the cancerous pancreas.

Embodiment 29. The method according to embodiment 28, further comprising treating the subject with a therapeutic regimen that inhibits one or more of fibroblast activation, the biologic activity of transforming growth factor (TGF) beta, or the biologic activity of alpha-v-beta-integrin in pancreatic stroma, thereby inducing the desmoplastic stroma to express a normal phenotype.

Embodiment 30. The method according to embodiment 28 or 29, wherein the step of detecting increased levels of active alpha-5-beta-1 integrin comprises contacting the isolated desmoplastic stroma with an antibody that specifically binds to an active conformation of alpha-5-beta-1 integrin.

Embodiment 31. The method according to embodiment 28 or 29, wherein the step of detecting decreased levels of active alpha-5-beta-1 integrin comprises contacting the isolated desmoplastic stroma with an antibody that specifically binds to an active conformation of alpha-5-beta-1 integrin.

Embodiment 32. The method according to embodiment 30 or 31, wherein the antibody is SNAKA51.

Embodiment 33. The method according to any one of embodiments 28 to 32, wherein isolating desmoplastic stroma from the pancreas of the subject comprises taking a biopsy of the pancreas.

Embodiment 34. The method according to embodiment 33, wherein the biopsy is a core needle biopsy.

Embodiment 35. The method according to embodiment 33, wherein the biopsy is a surgical biopsy.

Embodiment 36. A method, comprising isolating desmoplastic stroma from a cancerous kidney of a human subject, detecting increased levels of active alpha-5-beta-1 integrin localized away from three dimensional matrix adhesions, detecting increased focal adhesion kinase activity in the stroma, and administering to the subject an amount of interferon gamma effective to prime T lymphocytes; after a period of time following the administering of interferon gamma, isolating desmoplastic stroma from the kidney, detecting decreased levels of active alpha-5-beta-1 integrin localized away from three dimensional matrix adhesions, and detecting decreased focal adhesion kinase activity in the stroma, and administering to the subject an effective amount of an antibody that specifically binds to PD-1 or to PD-L1, thereby treating the cancerous kidney.

Embodiment 37. The method according to embodiment 36, further comprising treating the subject with a therapeutic regimen that inhibits one or more of fibroblast activation, the biologic activity of transforming growth factor (TGF) beta, or the biologic activity of alpha-v-beta-integrin in kidney stroma, thereby inducing the desmoplastic stroma to express a normal phenotype.

Embodiment 38. The method according to embodiment 36 or 37, wherein the step of detecting increased levels of active alpha-5-beta-1 integrin comprises contacting the isolated desmoplastic stroma with an antibody that specifically binds to an active conformation of alpha-5-beta-1 integrin.

Embodiment 39. The method according to embodiment 28 or 29, wherein the step of detecting decreased levels of active alpha-5-beta-1 integrin comprises contacting the isolated desmoplastic stroma with an antibody that specifically binds to an active conformation of alpha-5-beta-1 integrin.

Embodiment 40. The method according to embodiment 38 or 39, wherein the antibody that specifically binds to an active conformation of alpha-5-beta-1 integrin is SNAKA51.

Embodiment 41. The method according to any one of embodiment 36 to 40, wherein isolating desmoplastic stroma from the kidney of the subject comprises taking a biopsy of the kidney.

Embodiment 42. The method according to embodiment 41, wherein the biopsy is a core needle biopsy.

Embodiment 43. The method according to embodiment 41, wherein the biopsy is a surgical biopsy.

Embodiment 44. A method, comprising isolating a micro vesicle or exosome from the blood or ascites fluid of a human subject having kidney cancer, detecting increased levels active alpha-5-beta-1 integrin in the micro vesicle or exosome, and administering to the subject an amount of interferon gamma effective to prime T lymphocytes; after a period of time following the administering of interferon gamma, isolating a second micro vesicle or exosome from the blood or ascites fluid of a human subject, detecting decreased levels active alpha-5-beta-1 integrin in the micro vesicle or exosome, and administering to the subject an effective amount of an antibody that specifically binds to PD-1 or to PD-L1, thereby treating the kidney cancer.

Embodiment 45. The method according to embodiment 44, further comprising solubilizing the micro vesicle.

Embodiment 46. The method according to embodiment 44, further comprising solubilizing the exosome.

Embodiment 47. The method according to any one of embodiments 44 to 46, wherein the step of detecting increased levels active alpha-5-beta-1 integrin comprises contacting the internal contents of the micro vesicle or the exosome with an antibody that specifically binds to an active conformation of alpha-5-beta-1 integrin.

Embodiment 48. The method according to any one of embodiments 44 to 47, wherein the step of detecting decreased levels active alpha-5-beta-1 integrin comprises contacting the internal contents of the micro vesicle or the exosome with an antibody that specifically binds to an active conformation of alpha-5-beta-1 integrin.

Embodiment 49. The method according to embodiment 47 or 48, wherein the antibody that specifically binds to an active conformation of alpha-5-beta-1 integrin is SNAKA51.

Embodiment 50. A method, comprising isolating a micro vesicle or exosome from the blood or ascites fluid of a human subject having pancreatic cancer, detecting increased levels active alpha-5-beta-1 integrin in the micro vesicle or exosome, and administering to the subject an amount of interferon gamma effective to prime T lymphocytes; after a period of time following the administering of interferon gamma, isolating a second micro vesicle or exosome from the blood or ascites fluid of a human subject, detecting decreased levels active alpha-5-beta-1 integrin in the micro vesicle or exosome, and administering to the subject an effective amount of an antibody that specifically binds to PD-1 or to PD-L1, thereby treating the pancreatic cancer.

Embodiment 51. The method according to embodiment 50, further comprising solubilizing the micro vesicle.

Embodiment 52. The method according to embodiment 50, further comprising solubilizing the exosome.

Embodiment 53. The method according to any one of embodiments 50 to 52, wherein the step of detecting increased levels active alpha-5-beta-1 integrin comprises contacting the internal contents of the micro vesicle or the exosome with an antibody that specifically binds to an active conformation of alpha-5-beta-1 integrin.

Embodiment 54. The method according to any one of embodiments 50 to 53, wherein the step of detecting decreased levels active alpha-5-beta-1 integrin comprises contacting the internal contents of the micro vesicle or the exosome with an antibody that specifically binds to an active conformation of alpha-5-beta-1 integrin.

Embodiment 55. The method according to embodiment 53 or 54, wherein the antibody that specifically binds to an active conformation of alpha-5-beta-1 integrin is SNAKA51. The following examples are provided to describe numerous embodiments in greater detail. They are intended to illustrate, not to limit, the scope of the appended claims.

### EXAMPLES

### Example 1: Materials and Methods

Cell lines. Fibroblastic cells were cultured at 37° C. under 5% CO₂ using Dulbecco's Modified Eagle's Medium (Mediatech (Manasas, Va.)) supplemented with 10-15% (murine-human, respectively) Premium-Select Fetal Bovine Serum (Atlanta Biologicals (Lawrenceville, Ga.)), 2 mM L-Glutamine and 100 u/ml-μg/ml Penicillin-Streptomycin. Panc1, FAK^{+/+}, SYF and littermate wild type control cells were from the American Tissue Culture Collection (Manassas, Va.). FAK-KD and hTert controls were a gift.

Isolation and Characterization of fibroblastic cells. Pan-keratin-negative (AE1/AE3-Dako (Carpinteria, Calif.)) and vimentin-positive fibroblastic cells (EPR3776-Abcam (Cambridge, Mass.)) were isolated from fresh surgical normal and tumor tissue samples, and sorted as normal or activated. Cells were used for no longer than 12 passages while maintained within their own derived ECMs.

Preparation of fibroblasts-derived 3D ECMs. Extracellular matrices (ECMs) were obtained in the presence or absence of TGFβ1R small molecule inhibitor SB431542, DMSO, conformation-dependent anti-active α5 integrin functional antibody (SNAKA51), functional-blocking anti αvβ5 integrin (ALULA), or species matched non-immunized isotypic antibodies. Un-extracted 3D cultures were either processed for phenotypic characterization, ECM alignment measurements, or decellularized to assess maintenance of myofibroblastic phenotype.

ECM prompted myofibroblastic maintenance. Human naïve or assorted murine fibroblasts were incubated for 45 minutes prior to overnight culturing within assorted ECMs, prepared as above, in the presence or absence of 50 μg/ml SNAKA51, 60 μg/m ALULA, 250 μg/m mAb16, or BMAS diluted as instructed by the manufacturer (Millipore, Temecula, Calif.), IgG control, DMSO or small molecule inhibitors FAK (PF573,228) and SRC family kinases (PP2). ECM-induced phenotypes were assessed via RT-qPCR, Western Blot or immunofluorescence.

Confocal Image Acquisition. Confocal spinning disk Ultraview (Perkin-Elmer Life Sciences, Boston, Mass.) images were acquired with a 60×(1.45 PlanApo) oil immersion objective, under identical exposure conditions per channel using Velocity 6.3.0 (Perkin-Elmer Life Sciences, Boston, Mass.). Maximum reconstruction projections were obtained using MetaMorph 7.8.1.0 (Molecular Devices, Downingtown, Pa.).

ECM fiber orientation analysis. Fibronectin channel monochromatic images were analyzed via Imager OrientationJ plugin. Numerical outputs were normalized by setting mode angles to 0° and correcting angle spreads to fluctuate between −90° and 90°. Angle spreads for each experimental condition, corresponding to a minimum of three experimental repetitions and five image acquisitions per condition were plotted and their standard deviations calculated. The percentage of fibers oriented between −15° and 15° was determined for each normalized image-obtained data.

Bioinformatics and Statistics. Non-parametric Mann-Whitney tests were used to question experimental significances of all in vitro data. Significant p values in all figures were denoted by asterisks as follow: ****P<0.0001 extremely significant, ***P=0.0001-0.01 very significant, **P=0.01-0.05 significant and * P=0.06-0.10 marginally significant.

Regarding statistics pertinent to the TMAs, in order to identify clinical variables related to patient survival, univariate and multivariable analyses were performed by constructing decision trees using the Classification and Regression Trees (CART) methodology. Clinical variables were considered as predictors of survival time. The unified CART framework that embeds recursive binary partitioning into the theory of permutation tests was used. Significance testing procedures were applied to determine whether no significant association between any of the clinical variables and the response could be stated or whether the recursion would need to stop. No correction for multiple testing was employed, and a Type I error of 5% was used to test each hypothesis.

Smia Processes.

Conjugation of Q-dot to primary antibody. Q-dot antibody labeling kits were obtained from Molecular Probes-ThermoFisher Scientific. The SiteClick labeling kit constitutes of three step procedure lasting a few days and requires to have the desired antibody's carbohydrate domain modified to allow an azide molecule to be attached to it and in consequence DIBO-modified nanocrystals are linked to the antibody in question. Each conjugation was carried out as instructed by manufacturer provided protocols. Briefly, approximately 100 μg of antibody was used in each case. Unless there were excessive carrier proteins present in the antibody there was no need for antibody pre-purification step. The labeled antibodies were estimated at 1 μmolar concentration and stored in sterile conditions at 4° C. in light protected boxes. Sample incubations were all done at final concentration of ˜20 nMolar or 1:50 dilution.

Immunolabeling of FFPE tissue sections. Slides were exposed to short wave UV lamp for about 30 minutes in light protected box to quench auto fluorescence. They were then kept in a light protected (i.e., dark) box until used. Sections were deparaffinized in xylene and rehydrated in ascending graded alcohol to water dilutions. Sections were then treated with Digest-All (Invitrogen) and permeablized in 0.5% TRITON® X-100 non-ionic surfactant. After treating them with blocking buffer as in vitro samples were first incubated with the above-mentioned Q-dot pre-labeled antibodies (i.e., Q655 SNAKA51, Q565 mAb11) overnight at 4° C. Sections were washed as in vitro and incubated with a mix of mouse monoclonal “cocktail” containing anti-pan-cytokeratin (clones AE1/AE3, DAKO), anti-EpCam (MOC-31EpCam, Novus Biologicals (Littleton, Colo.)) and anti-CD-70 (113-16 Biolegend (San Diego, Calif.)), to detect epithelial/tumoral locations, together with rabbit monoclonal anti-vimentin (EPR3776, Abcam) antibodies (mesenchymal stromal components) for 2 hours at room temp. Pre-incubated Q-dot labeled antibodies could no longer be recognized by secondary antibodies thus allowing for indirect immunofluorescent detection of tumoral and stromal masks. Secondary antibodies were as in vitro and included donkey anti-mouse Cy2 and donkey anti-rabbit Cy3. Nuclei were stain using Draq-5 (as in vitro). Sections were quickly dehydrated in graded alcohol and clarified in Toludene before mounting in Cytoseal-60. Slides were cured overnight at room temperature before the imaging.

Image acquisitions of FFPE fluorescently labeled sections. FFPE sections of biological samples are known to give strong broad autofluorescence thus obscuring the specific (labeled) fluorescent signal. To overcome this problem, images were collected using Caliper's multispectral imaging system (PerkinElmer) which utilizes a unique imaging module with tunable Liquid Crystal. Two different systems namely Nuance-FX (for 40× objective) and Vectra (for 20× objective and high throughput) were used depending on the acquisition needs. A wavelength length based spectral library for each system and each tissue type (i.e., pancreas and kidney) was created by staining control sections with individual fluorophores or mock treating samples to including the specific autofluorescence spectra. Once a spectral library was constructed per organ type it was saved and used for the subsequent image acquisition and analysis. All excitations were achieved via high intensity mercury lamp using the following filters (emission-excitation): for Nuance, DAPI (450-720), FITC (500-720), TRITC (580-720), CY5 (680-720); for Vectra, DAPI (440-680), FITC (500-680), TRITC (570-690), CY5 (680-720). For emissions collection: “DAPI” filter (wavelength range 450-720) was used for all Q-dot labeled markers, while masks used the conventional FITC, TRITC and CY5 filters. After collecting all image cubes (including all channels) images were unmixed to obtain 16 bit greyscale individual stains monochromatic files. Using Photoshop's Levels and Batch Conversion Functions the images were processed in bulk to render identically scaled 8 bit monochromatic images per channel. The resulting images were sampled to set identical threshold values for each channel which were used to feed the values needed for analyses in SMIA-CUKIE signifying positive labeled pixels.

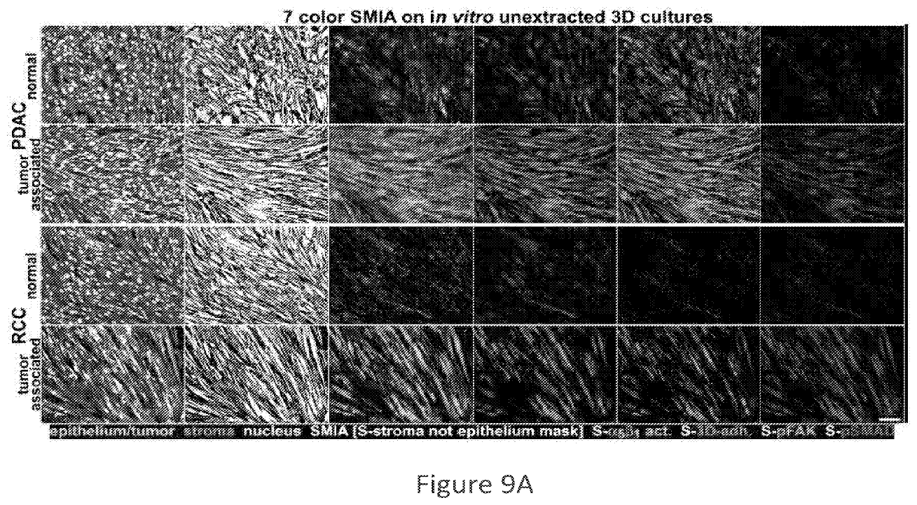

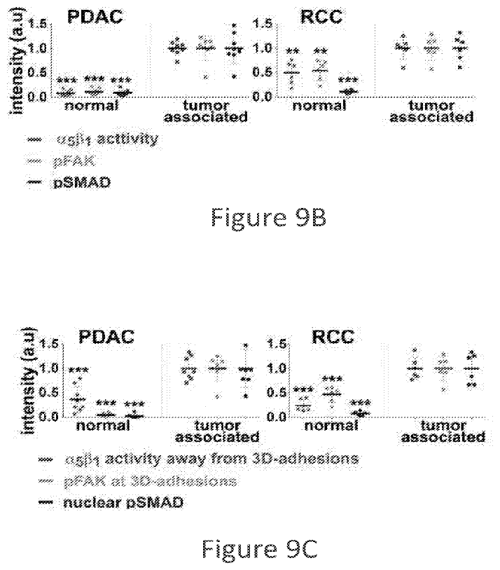

FIG. 9A shows SMIA analysis of in vitro PDAC (top) and RCC (bottom), normal and desmoplastic fibroblasts. Right most panels demonstrate the positive staining of vimentin, and lack of cytokeratin, indicating the purity of the fibroblasts isolated, nuclei are marked. White (SMIA) masks in the next column, represent vimentin-covered area alone as recognized by the software (analogous to stroma in vivo). Next panels show are the assorted SMIA-positive pixels of the listed markers corresponding to: active α5β1 integrin, 3D-adhesions, pFAK, and pSMAD. Scale bar represents 50 μm. FIG. 9B shows graphs summarizing normalized mean total intensity levels of active α5β1 integrin (left bullets), pFAK (center bullets) and pSMAD (right bullets) from similar samples as in FIG. 9A. Graphs in FIG. 9C show intricate analysis of samples also from FIG. 9A, displaying mean intensity of active α5β1 integrin distributed away from 3D-adhesions (left bullets), pFAK at 3D-adhesions (center bullets), or pSMAD at nuclear compartments (right bullets).

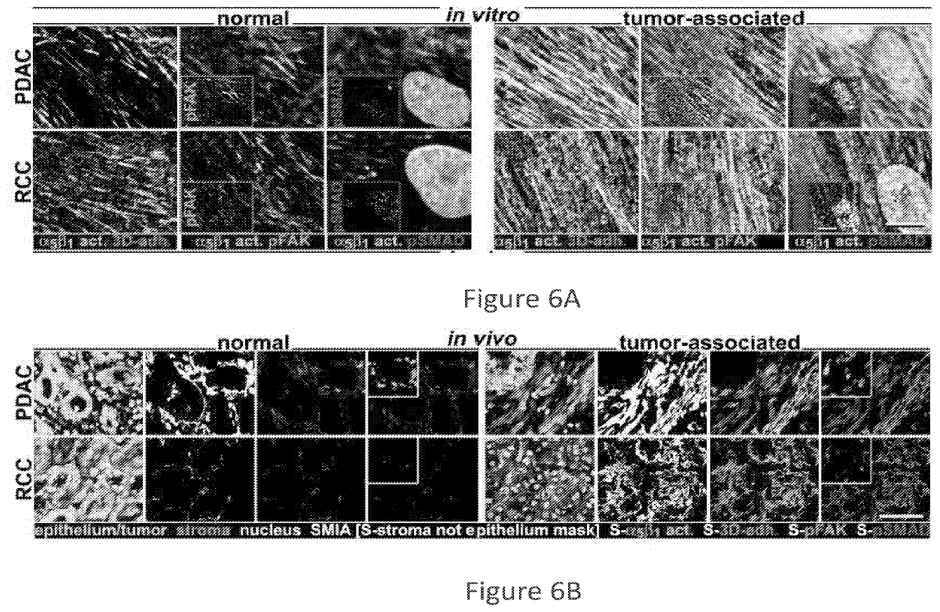

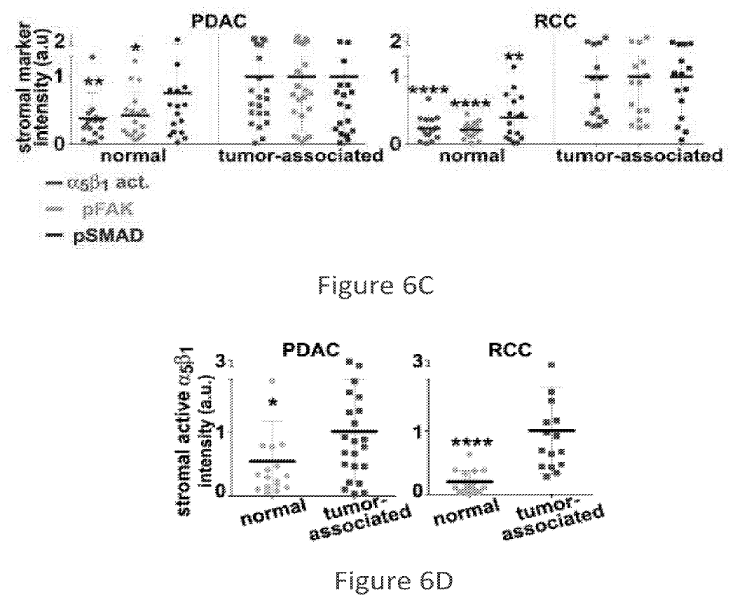

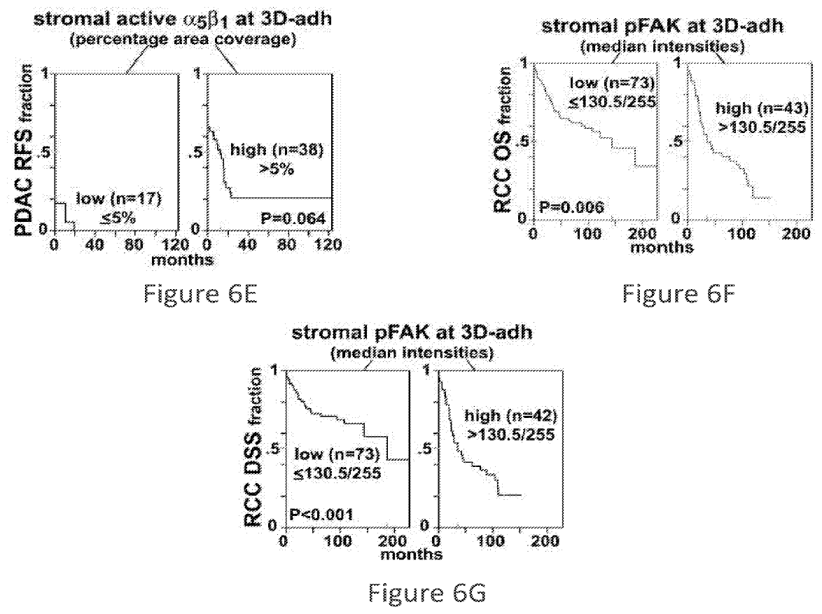

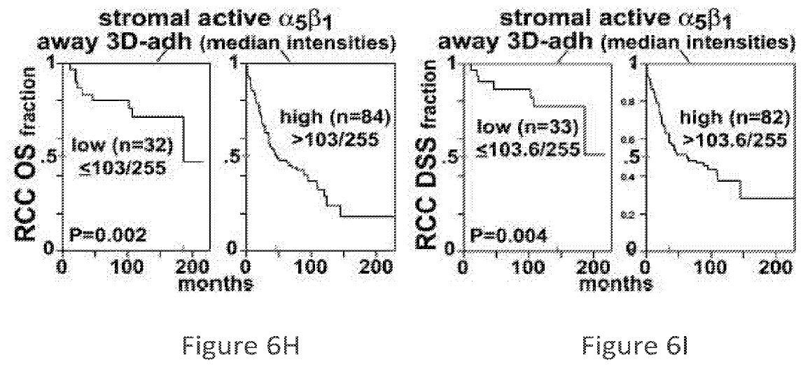

SMIA-CUKIE usage and outputs. SMIA-CUKIE was written for the bulk analysis of high throughput acquired monochromatic images corresponding to the simultaneously labeled channels. See, Franco-Barraza et al., eLife, 2017 (6: e20600 DOI: 10.7554/eLife.20600). As an example, masks in FIG. 6B and FIGS. 9A and 9B were generated using SMIA-CUKIE and demonstrate how the software isolates stromal locations while omitting tumoral positive areas, based on mask value thresholds provided by the user.

Images were sorted in “Batch Folders” each containing the five monochromatic images corresponding to the original (unmixed) sample. The new written software, (github.com/cukie/SMIA_CUKIE), was created to bulk process and analyze batches of monochromatic images providing localization (masks), intensities, and similar quantifying values (markers), including co-localizations of multichannel monochromatic immunofluorescent (or IHC, etc.) images. The software can identify intersection areas between an unlimited amount of masks while queried marker values and locations can also be estimated for numerous interrogations. The software requires identification of common nomenclatures to recognize each type of monochromatic image (vim for vimentin etc.) and necessitates information regarding the available number of masks and markers deposited in the batch folders containing the images (in our case there were three masks corresponding to nuclei, epithelium/tumor and stroma as well as two markers corresponding to adhesion structures and activated integrin). For each of the masks and markers the software requires a threshold number, which indicated the value (0-255) of pixel intensity that is to be considered positive for each channel (corresponding to each mask and marker). The software then allows choosing amongst all possible query combinations or provides an option for the user to write the desired tests to be queried. For example, when integrin activity values were requested at bona fide stromal locations, the software was instructed to look for “SNAKA under vimentin, NOTepi/tumor”. In this example “SNAKA” was the nomenclature we used for the active integrin channel while vimentin and epi/tumor served as masks. After running this function the software rendered an excel file containing values of area coverage for the marker at the mask intersection (bona fide stroma) as well as total area coverage related to the image. Similarly, values included mean medium, standard deviation, total intensity and integrated intensities. The software's output includes the name of the folder batch each query is related to. In addition, the software can be instructed to provide image outputs corresponding to requested mask locations as well as markers shown solely at the corresponding mask intersections (as shown in FIGS. 6B and 9A).

Exosome Purification. Cells are plated and cultured to confluence in full supplement media. Cells are washed with PBS and cultured in serum-free high glucose DMEM supplemented with 10% Lipid (exosome) depleted media and 1% pen/strep for 48 hours, and supplemented with 50 mg/ml ascorbic acid. Media is collected after 48 hours andmaintained cold (4° C.). Conditioned media (CM) is centrifuged at 2.5×1000 RPM and supernatant is collected and filtered (0.22 micron filter). Filtered conditioned media is concentrated and ultracentrifuged at 34.0×1000 RPM (100,000×G) for 2 hours. Precipitate (exosomal collection) is washed with sterile PBS and additionally spun down again at 34.0×1000 RPM, and pellet is collected.

CRISPR/CAS9 mediated knockout (KO) of/35 integrin in fibroblasts. Immortalization of human fibroblasts. Before performing CRISPR/Cas9 mediated KO of (35 integrin, fibroblasts were immortalized with human telomerase (hTERT), using a retroviral infection with the pBABE-neo-hTERT vector, which was a gift from Robert Weinberg (Addgene plasmid #1774). First, to produce functional retrovirus, packaging cells Phoenix-Amphotropic (φNX) (ATCC #CRL-3213) at 50% confluence in 10 cm culture dishes were transfected with 10 μg of pBABE-neo-hTERT vector using 10 μL of Lipofectamine (Invitrogen, #18324) and 40 μL of Plus Reagent (Invitrogen, #11514015) in 6 mL of serum/antibiotics free Opti-MEM (Gibco, #31985062) culture media overnight at 37° C. in a cell culture incubator. The following morning, media was replaced with 10 mL of fresh Opti-MEM (serum/antibiotics free) and incubated 24 hours at 32° C. In the morning of days 2, 3 and 4 post-transfection, the conditioned media containing retrovirus was collected and filtered through a 0.45 μm syringe filter (Millipore, #SLHVO13SL) and used for subsequent retroviral transfections of fibroblasts. For the viral infection, fibroblasts were cultured in 10 mL of conditioned media containing the retrovirus (supplemented with 4 μg/mL Polybrene (Santa Cruz, #sc-134220)) for 8 hours at 37° C. Then cells were washed and incubated with fresh culture media (DMEM 10% FBS, 1% L-Glut, 1% P/S) and incubated at 37° C. overnight. Beginning the next day, this infection procedure was repeated two additional times, for a total of three retroviral infections. Fibroblasts were then selected with G-418 at a concentration of 750 μg/mL until cells grew back to—90% confluence. Cells were then expanded and tested for over-expression of hTERT, and lack of p16, by western blotting to confirm hTERT overexpression. These cells were used for CRIPSR/CAS9 mediated knockout of (35 integrin.

gRNA Design. To knockout the 135 integrin subunit encoding gene from fibroblasts, CRISPR/CAS9 gene editing was performed to introduce a frameshift mutation that disrupts the reading frame causing a premature stop codon to be read, ultimately halting translation and knocking out this gene.

First, gRNAs specific for 135 integrin were designed by targeting exon 3. The first 200 base pairs of exon 3 were inserted into MIT Optimized CRISPR Design website (crispr.mit.edu/) and the top two scoring gRNAs were selected, based on the presence of a PAM site (CAS9 recognition site) and limiting off-target binding.

The two independent gRNA sequences were as follows:

gRNA 1:
(SEQ ID NO: 1)
ACCGAGAGGTGATGGACCGT;
and
gRNA 2:
(SEQ ID NO: 2)
CACCGAGAGGTGATGGACCG

For a non-targeting gRNA control, the following gRNA against eGFP was designed:

(SEQ ID NO: 3)
CATGTGATCGCGCTTCTCGT.

Generation of Lentiviral KO Vector. Once the gRNA sequences were designed and ordered (Integrated DNA Technologies), they were cloned into the LentiCRISPR v2 vector (LentiCRISPR v2 was a gift from Feng Zhang; Addgene plasmid #52961). This is a dual-expression vector, expressing the CRISPR/CAS9 protein, as well as the cloned gRNA sequence driven by the human U6 promoter. Briefly, DNA oligos representing the gRNA sequences are listed below, with overhangs compatible with the Esp3I restriction enzyme (bold, underlined) and an additional G added to the beginning of each gRNA, with a complementary C on the reverse oligo (bold, italics), for efficient transcription driven by the human U6 promoter.

Integrin 35 gRNA 1.1:
(SEQ ID NO: 4)
CACCGACCGAGAGGTGATGGACCGT
Integrin 85 gRNA 1.2:
(SEQ ID NO: 5)
AAACACGGTCCATCACCTCTCGGTC
Integrin 35 gRNA 2.1:
(SEQ ID NO: 6)
CACCGCACCGAGAGGTGATGGACCG
Integrin 35 gRNA 2.2:
(SEQ ID NO: 7)
AAACCGGTCCATCACCTCTCGGTGC
eGFP gRNA 1.1:
(SEQ ID NO: 8)
CACCGCATGTGATCGCGCTTCTCGT
eGFP gRNA 1.2:
(SEQ ID NO: 9)
AAACACGAGAAGCGCGATCACATGC.

Next, the gRNA oligos stocks were diluted to 100 μM, and 1 μL of each gRNA pair was added to a T4 PNK reaction mixture (NEB, #M0201S) for a 10 μl reaction. The reaction was allowed to run in a thermal cycler to phosphorylate and anneal the oligos, according to the following program:
1) 37° C. for 30 minutes;
2) 95° C. for 5 minutes; and
3) Decrease 5° C. every minutes until 25° C.

To prepare the vector for cloning, 5 μg of LentiCrispr v2 vector were simultaneously cut with Fast Digest Esp3I (ThermoFisher, #FD0454) and dephosphorylated with Fast AP (ThermoFisher, #EF0651) for 30 minutes at 37° C. and subsequently run on a 1.5% agarose gel and purified for cloning using the GeneJet Gel Extraction Kit (Thermofisher, #K0691). To clone the annealed gRNA oligos into the vector, the oligos were diluted 1:200 in RNAase Free water (ThermoFisher, #4387937), and added to the quick ligation reaction mixture (NEB, #M2200S), along with 1 μL of the digested and dephosphorylated vector, and the reaction was carried out for 10 minutes at room temperature.

For bacterial transformation, 2 μL of the reaction was mixed with 50 μL of competent Stb13 strain of E coli for 30 minutes on ice. The bacteria were then heat shocked for 45 seconds at 42° C. and immediately transferred to ice for 2 minutes. Then 50 μL of the transformed bacteria were spread onto an LB/agar dish containing 100 μg/mL ampicillin and incubated at 37° C. overnight.

The next day, single colonies were screened by colony PCR using the U6 promoter Forward primer: GAGGGCCTATTTCCCATGATT (SEQ ID NO:10) and the corresponding reverse gRNA primers (gRNA x.2). Positive clones were selected to expand for plasmid purification and sequencing.

CRISPR Lentivirus Production. For functional Lentiviral production, viruses were generated in 293T cells using the cloned LentiCrispr v2 plasmids, and 2 packaging plasmids: psPAX2 (psPAX2 was a gift from Didier Trono; Addgene plasmid #12260), and VSVg. Briefly, 10 ng of cloned LentiCrispr V2, 5 μg of psPAX2, and 2 μg of VSVg were mixed in 1 mL serum-free/antibiotic-free DMEM in an 1.5 mL Eppendorf tube. 30 μl of X-treme Gene 9 (Roche, #06365787001) was added to the DNA mixture and gently mixed with the pipette tip. The mixture was allowed to sit for 45 minutes at room temperature. Next, the mixture was added drop-wise to a T75 flask containing 5 mL serum-free/antibiotic-free DMEM and 293T cells at ˜85% confluence and slowly rocked for 30 seconds to evenly mix the DNA transfection mixture. The next morning, the serum free media was removed from the 293T cell flasks, and 10 mL of fresh DMEM containing 10% FBS and 1% penicillin/streptomycin were added to each flask. 2 days and 4 days post-transfection, the media was collected and filtered through a 0.45 μM syringe filter (Millipore, #SLHVO13SL) and was used immediately for viral infection of target cells, or stored at −80° C. until needed.

CRISPR Lentiviral Infection. Target cells (naïve or desmoplastic fibroblasts) were seeded at ˜40% confluence in 2 mL complete fibroblast media in a 6 well plate. The following day, the target cells were infected with 2 mL of lentivirus for each corresponding CRISPR construct (eGFP or integrin gRNAs) in the presence of 10 μg/mL Polybrene (Santa Cruz, #sc-134220). As a control for the infection, cells were infected with a lentivirus overexpressing GFP, and the appearance of GFP-positive cells signified a successful infection. About 24 hours later, the media was replenished with fresh complete media for each cell type. After about 72 hours, puromycin selection (1 μg/mL for naïve fibroblasts, 2 μg/mL for desmoplastic fibroblasts) of the infected cells began. The selection process lasted between 7-10 days; cells were expanded, and the efficiency of CRISPR/CAS9 knockout was assessed by western blotting. The cell lines with the greatest degree of target protein knockout were used for subsequent experiments.

### Example 2: TGFβ is Necessary for Functional Desmoplastic Extracellular Matrix (ECM) Production

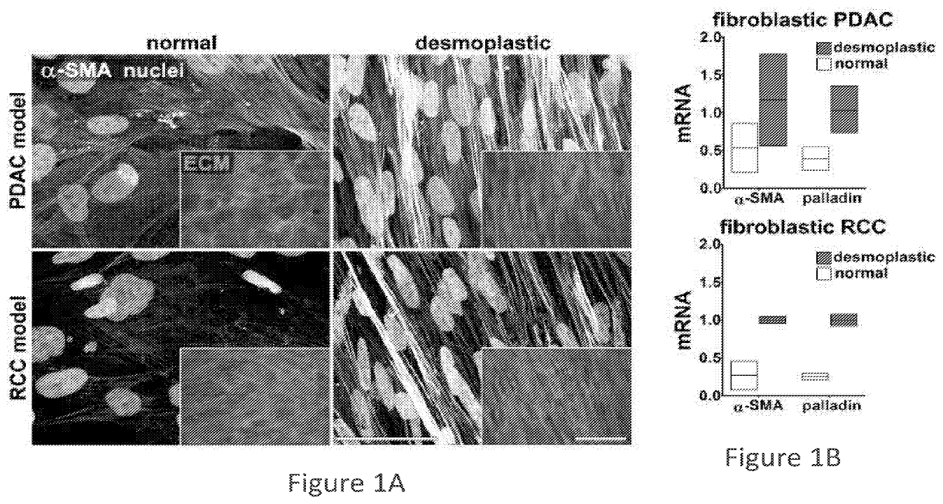

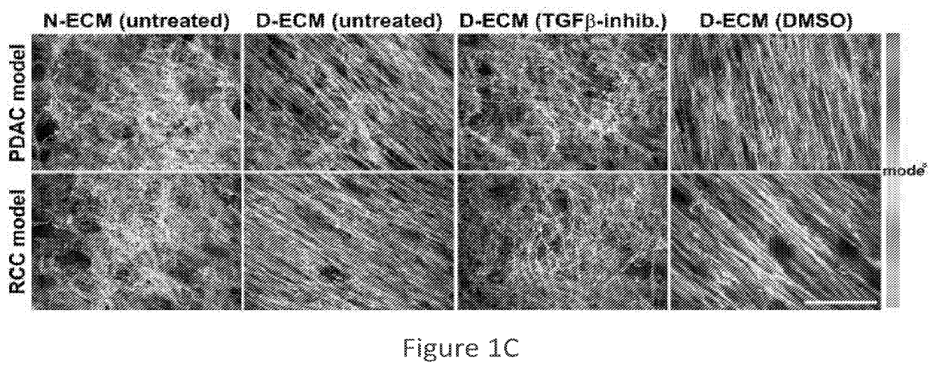

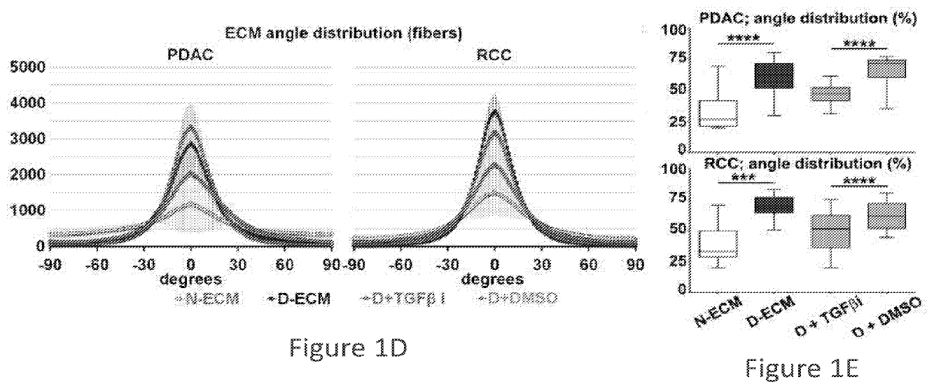

FIGS. 1A through 1E show desmoplastic (D)-ECM remodeling by TGFβ-blockage bestows normal (N)-ECM-like phenotypes. Fibroblasts were isolated from pancreatic ductal adenocarcinoma (PDAC) and renal cell carcinoma (RCC) surgical samples and their ECM producing phenotypes were assessed prior to ECM decellularization via fibroblastic extraction. FIG. 1A shows representative images of normal vs. desmoplastic fibroblastic phenotypes, subsequent to ECM production, are shown in magenta (inserts). Complementary low/heterogeneous vs. high/organized α-SMA levels, with heterogeneous/round vs. elongated/spindled cell nuclei, and disorganized/woven vs. parallel/aligned ECM fibers are evident in the representative images. FIG. 1B shows normal vs. desmoplastic mRNAs levels, corresponding to fibrosis markers, α-SMA and palladin obtained via RT-qPCR from the indicated 3D-cultures following ECM production. FIG. 1C shows images representative of cell-derived ECM fibers phenotypes (organized vs. disorganized) from normal (N-ECM), desmoplastic (D-ECM), and TGFβ blocked (TGFβ-inhib.) or vehicle treated (DMSO) during D-ECM production. FIG. 1D shows curves corresponding to the indicated experimental conditions (as in FIG. 1C) depicting average and variations of ECM fibers angle distributions that were normalized to cero degree modes. FIG. 1E shows graphs corresponding to plotted data depicting summarized percentages of ECM fibers distributed at 15 degree angles from the mode corresponding to the indicated experimental conditions. Scale bars represent 50 μm. Comparisons between N- and D-ECMs rendered statistical significant differences with, P values smaller than 0.0001, in both models while TGFβ blockage during D-ECM production established that alignments were reduced to 47% (P<0.0001; n=21) in PDAC and 51% (P=0.0028; n=26) in RCC rendering ECMs disorganized.

Pancreatic and renal surgical sample-isolated fibroblastic cells were characterized as normal/naïve or desmoplastic/activated by assessing expression of the desmoplastic markers palladin and smooth muscle alpha-actin (α-SMA) (FIGS. 1A, 1B, and 6A). Their corresponding normal or activated phenotypes were confirmed using unextracted three-dimensional cultures subsequent to fibroblastic ECMs production. Results presented in FIG. 1A show that desmoplastic pancreatic ductal adenocarcinoma (PDA) and renal cell carcinoma (RCC) cells has characteristic spindled nuclei, increased levels of stress-fiber localized α-SMA, and anisotropic ECM fibers. Quantification of matrix alignment (see, FIGS. 1C-1E) showed that 62% and 68% of total detected fibers were distributed at 15° from the mode angle in D-ECMs in PDAC and RCC cultures, while angle distributions of normal ECMs (N-ECMs) were only 27% and 33%, respectively. ECMs with a minimum median of 55% of fibers oriented within 15° from the mode distribution angle were considered anisotropic in subsequent mechanistic studies.

FIGS. 6A through 6I show in vivo validations of in vitro uncovered phenotypes suggesting that levels and distributions of active α5β1 integrin and pFAK can effectively predict human PDAC and RCC recurrences. FIG. 6A depicts in vitro characterization of normal vs. tumor-associated (desmoplastic) fibroblasts in unextracted 3D cultures. Confocal-acquired images of indirect immunofluorescence of desmoplastic markers from PDAC and RCC models, are shown. Markers are shown overlaid to depict the distribution of active α5β1 integrin with regards to 3D-adhesion locations, p-FAKY³⁹⁷, pSMAD⅔, and nucleus. Inserts correspond to monochromatic marker images. Scale bars; 10 μm.

FIG. 6B shows seven simultaneous multi-channel immunofluorescent (SMI) approach implemented to FFPE surgical samples corresponding to the original patients from where fibroblasts used in vitro were harvested (see, FIGS. 6B-6C) and to the well-annotated cohorts of PDAC (see, FIG. 6E) and RCC (see, FIGS. 6F-6I) patients that were retrospectively collected in house. In FIG. 6B, left most panels are overlaid images including the three monochromatic channels used for “masked” locations; epithelial/tumoral areas, stromal vimentin, and nucleus are shown. The SMIA-CUKIE software was instructed to render an intersection “mask” image (SMIA-mask “S”; white) corresponding to pixel areas selected as stroma positive and epithelial/tumoral negative to exclude potential mesenchymal to epithelial-transduced tumoral locations. Next, to the right of the SMIA mask image, are images of the corresponding only to bona fide stromal pixel overlaid “markers” showing active α5β1 integrin (S-α5β1 act) and 3D-adhesions (S-3D-adh.). The right panels are images corresponding to the same SMIA selected pixels depicting overlaid p-FAKY (S-pFAK) and pSMAD⅔ (S-pSMAD). Inserts correspond to stromal selected areas showing pSMAD-nuclei overlays. Scale bar; 50 μm. Only pixels that were selected by the software as stroma positive and epithelial/tumoral negative areas (corresponding to white SMIA-mask) are shown in the three right panels, as these were the ones that were quantitatively analyzed.

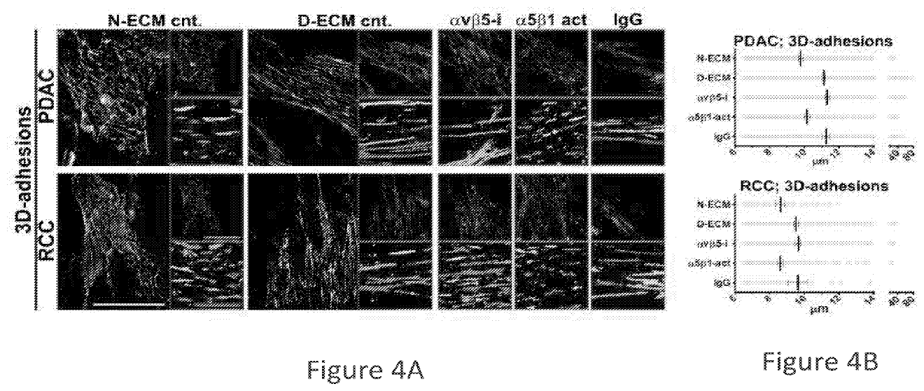

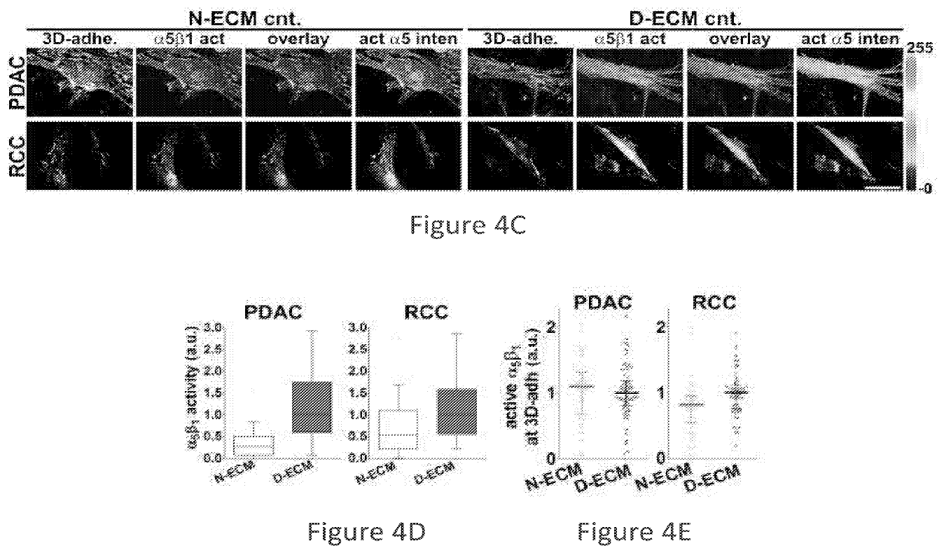

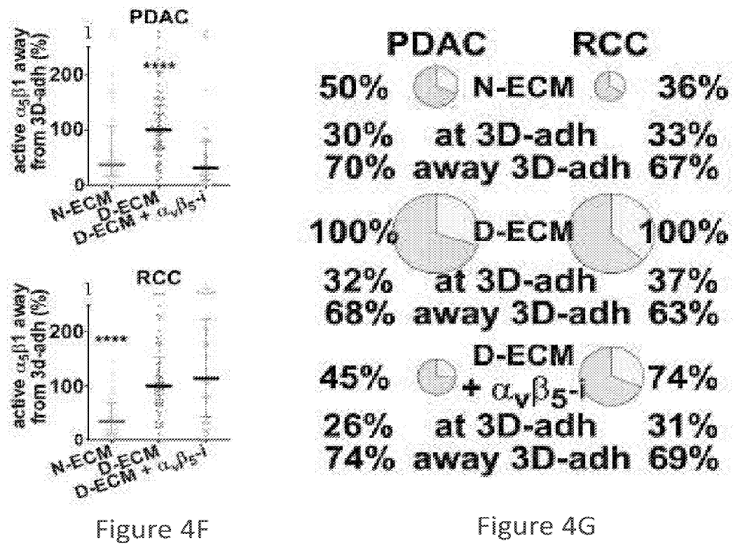

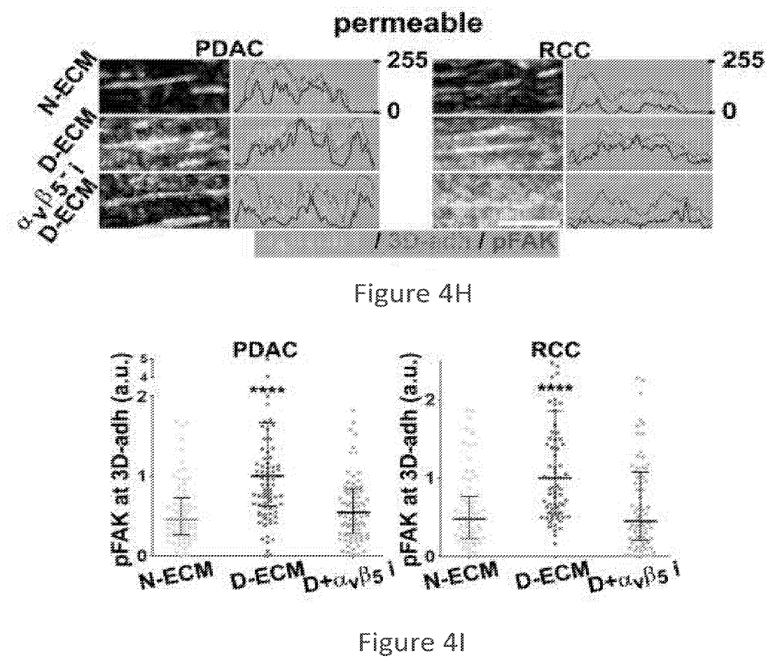

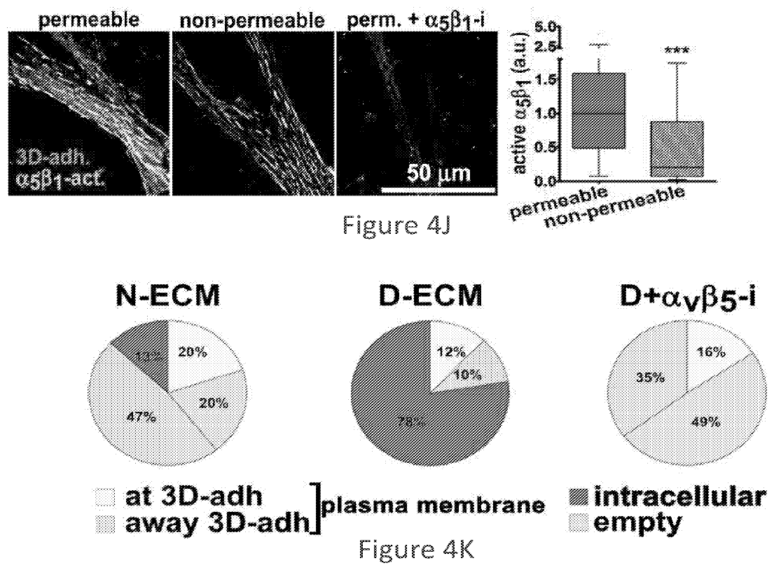

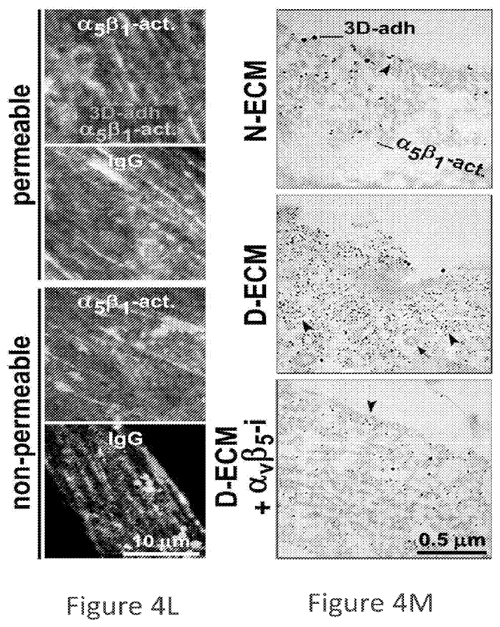

Individual patient SMIA-CUKIE generated intensity values were used for plotting the graphs in FIG. 6C representing stromal levels of active α5β1 integrin (left-most bullets), p-FAK-Y³⁹⁷ (center bullets) and pSMAD⅔ (right-most bullets); and active α5β1 integrin away from 3D-adhesions in FIG. 6D, which validate results obtained in vitro (compare to FIG. 4G). Overlay pixels that appear in white depict areas of co-localized masks. Significance asterisks represent the following: C, PDAC*** P=0.0013,** P=0.0371; RCC****P<0.0001, ***P=0.0031. D,** P=0.0371 and**** P<0.0001.

SMIA-CUKIE generated values of the same seven colors corresponding to human cohort constructed TMAs (listed in Table 2) were analyzed, together with patient collected survival data, and used for the CART generated survival curves shown in E-I. E; RFS curves of PDAC patients representing low (left) and high (right) stromal area coverage percentages of active α5β1 integrin localized at 3D-adhesions. FIGS. 6F and 6G, respectively, correspond to OS and DSS survival curves representing RCC patients presenting low (left) and high (right) median values of intratumoral stroma pFAK localized at 3D-adhesion. FIGS. 6H and 61, respectively, correspond to OS and DSS survival curves of RCC patients presenting low (left) and high (right) active α5β1 integrin median intensity values localized at stromal locations away from 3D adhesions. Tick-lines crossing Y axes represent 0.5 survival marks and correspond to X axes locations that mark the median survival times obtained from the assorted curves. P values are shown.

Formation of anisotropic fibers was TGFβ-dependent, based on experiments using inhibition of the TGFβ1 receptor with SB-431542. Blockage of TGFβ signaling during D-ECM production resulted in a fiber alignment phenotype reminiscent of N-ECM (36% in PDAC and 47% in RCC; see, FIGS. 1C-1D), while it prevented de novo N-ECM assembly (see, FIG. 6B). Assessment of TGFβ levels that are stored within ECMs in these cultures showed—44% (P=0.0014) higher TGFβ levels in D-ECMs, compared to N-ECMs and—60% (<0.0001) reduction if SB-431542 was used during D-ECM production (see, FIG. 6C).

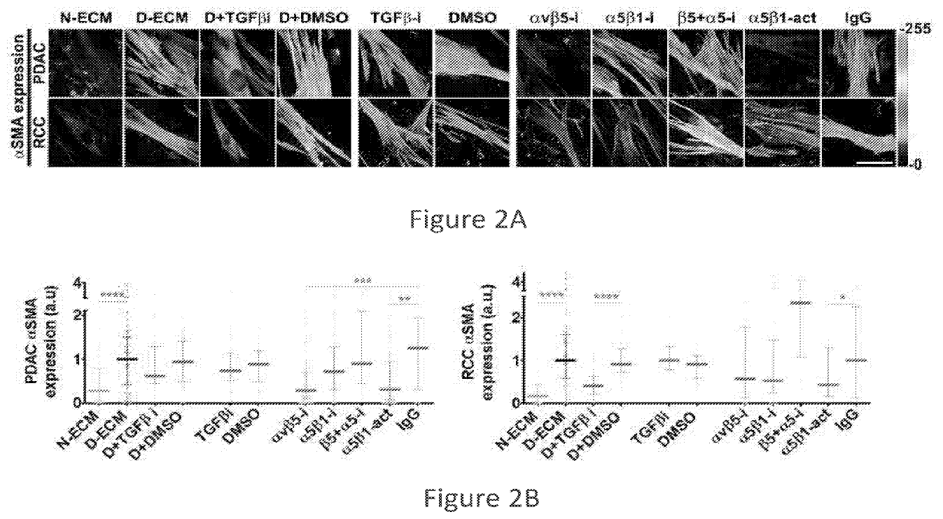

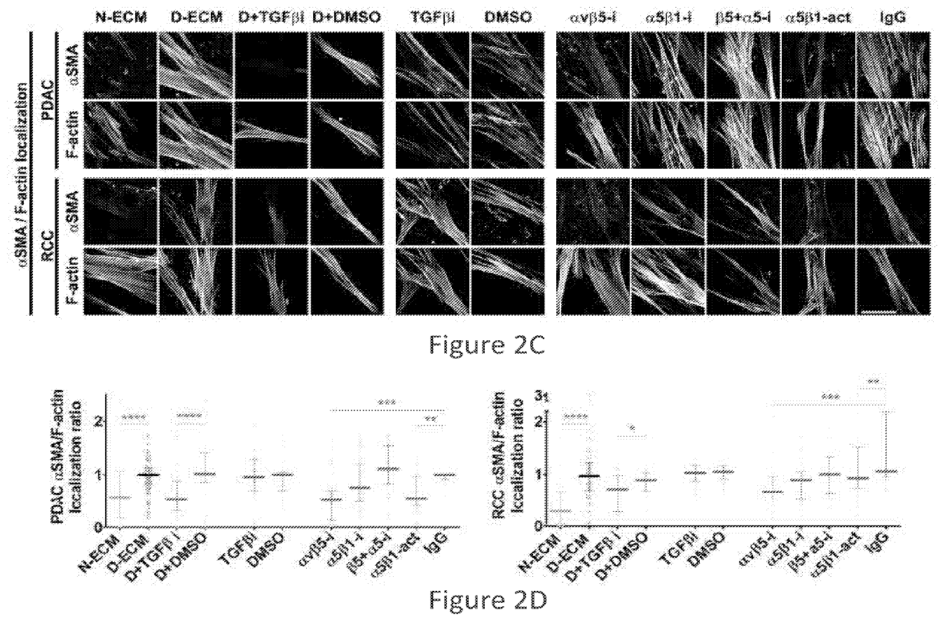

Stripping of matrix-producing cells from their secreted ECMs renders discrete “extracted” matrix substrates. The residual three-dimensional (3D) D-ECM has been shown capable of inducing a myofibroblastic phenotype in naïve fibroblasts. The assorted extracted N and D-ECM, as well as D-ECMs that were produced in the presence of SB-431542, were re-seeded with naïve normal pancreatic or renal fibroblasts. Next, we tested the ability of these ECMs to induce a myofibroblastic phenotype de novo. N-ECM and SB-431542-treated D-ECM triggered comparable phenotypes while vehicle control-treated and intact D-ECMs effectively induced a myofibroblastic phenotype (see, Table 1). Fibroblasts cultured in N-ECM from pancreatic and renal cells expressed 27% and 17% of the α-SMA protein expressed in fibroblasts cultured in D-ECM from PDAC and RCC (see, FIGS. 2A and 2B). Both N-ECM and D-ECM produced under TGFβ blockage also produced limited stress fiber localization of α-SMA in naïve fibroblasts, relative to the untreated D-ECM (58% and 55%) in the PDAC models, with a significant but lesser effect in RCC (29% and 72%) (see Table 1, FIGS. 2C and 2D). Differences in α-SMA reflected a post-transcriptional effect, as α-SMA mRNA levels were comparable in all growth conditions (see, FIG. 6D). Performance of similar experiments in the presence of the protein synthesis inhibitor cycloheximide also did not influence outcomes (see, FIG. 6E), suggesting the main function of D-ECM is to enhance α-SMA recruitment to or retention at stress fibers, extending protein half-life.

It was next determined if TGFβ activity is also necessary for myofibroblastic activation in naïve pancreatic and renal fibroblasts plated in TAF-derived D-ECMs from both the PDAC and RCC models. Relative to untreated conditions, vehicle control treated pancreatic and renal naïve fibroblasts plated in D-ECMs had α-SMA expression levels of 94% and 93%, respectively, while plating these cells in D-ECM in the presence of the TGFβ inhibitor SB-431542 did not significantly reduce expression (62% and 100%; P=0.34 and P=0.15) (see, FIGS. 2A and 2B and Table 1). Similarly, stress fiber localization of α-SMA was comparable in both PDAC and RCC models when cells were replated using vehicle-treated versus TGFβ-inhibited conditions (see, FIGS. 2C and 2D and Tablet). These results suggested that TGFβ inhibition during DECM production reduces the ability of cells to produce ECM that can induce myofibroblastic activation, but that once D-ECM has formed, TGFβ is subsequently dispensable.

TABLE 1
Levels and significance values of α-SMA expression and stress fiber localization

| PDAC | | | | | | | | | | | |
|---|---|---|---|---|---|---|---|---|---|---|---|
| α-SMA | N- | D- | D + | D + | | | | | | α5β1- | |
| expression | ECM | ECM | TGFβi | DMSO | TGFβi | DMSO | αvβ5-i | α5β1-i | β5 + α5-i | act | IgG |
| 25% | 0.05 | 0.41 | 0.52 | 0.48 | 0.44 | 0.49 | 0.11 | 0.31 | 0.44 | 0.08 | 0.31 |
| Percentile | | | | | | | | | | | |
| Median | 0.27 | 1.00 | 0.73 | 0.89 | 0.62 | 0.94 | 0.29 | 0.71 | 0.90 | 0.32 | 1.25 |
| 75% | 0.78 | 1.50 | 1.14 | 1.19 | 1.30 | 1.41 | 0.70 | 1.27 | 2.09 | 0.96 | 1.95 |
| Percentile | | | | | | | | | | | |
| N-ECM | | P < | P = | P < | P = | P < | P = | P < | P = | P = | P = |
| | | 0.0001 | 0.0002 | 0.0001 | 0.0010 | 0.0001 | 0.7136 | 0.0001 | 0.0007 | 0.4913 | 0.0013 |
| D-ECM | | | P = | P = | P = | P = | P < | P = | P = | P < | P = |
| | | | 0.0198 | 0.1208 | 0.3401 | 0.7408 | 0.0001 | 0.1701 | 0.5018 | 0.0001 | 0.3006 |
| D + TGFβi | | | | P = | P = | P = | P < | P = | P = | P = | P = |
| | | | | 0.6236 | 0.7499 | 0.2904 | 0.0001 | 0.7309 | 0.3837 | 0.0014 | 0.0735 |
| D + DMSO | | | | | P = | P = | P < | P = | P = | P = | P = |
| | | | | | 0.6430 | 0.5125 | 0.0001 | 0.4473 | 0.4137 | 0.0008 | 0.1223 |
| TGFβi | | | | | | P = | P = | P = | P = | P = | P = |
| | | | | | | 0.3361 | 0.0003 | 0.9501 | 0.3620 | 0.0072 | 0.1370 |
| DMSO | | | | | | | P < | P = | P = | P = | P = |
| | | | | | | | 0.0001 | 0.2688 | 0.5509 | 0.0004 | 0.1307 |
| αvβ5-i | | | | | | | | P < | P = | P = | P = |
| | | | | | | | | 0.0001 | 0.0004 | 0.6414 | 0.0002 |
| α5β1-i | | | | | | | | | P = | P = | P = |
| | | | | | | | | | 0.2192 | 0.0003 | 0.1229 |
| β5 + α5-i | | | | | | | | | | P = | P = |
| | | | | | | | | | | 0.0026 | 0.9171 |
| α5β1-act | | | | | | | | | | | P = |
| | | | | | | | | | | | 0.0020 |
| IgG | | | | | | | | | | | |
| RCC | | | | | | | | | | | |
| α-SMA | N- | D- | D + | D + | | | | | | α5β1- | |
| expression | ECM | ECM | TGFβi | DMSO | TGFβi | DMSO | αvβ5-i | α5β1-i | β5 + α5-i | act | IgG |
| 25% | 0.03 | 0.58 | 0.21 | 0.73 | 0.78 | 0.57 | 0.13 | 0.23 | 1.09 | 0.17 | 0.14 |
| Percentile | | | | | | | | | | | |
| Median | 0.17 | 1.00 | 0.41 | 0.92 | 1.00 | 0.93 | 0.58 | 0.53 | 2.35 | 0.43 | 1.00 |
| 75% | 0.43 | 1.61 | 0.64 | 1.28 | 1.33 | 1.12 | 1.79 | 1.48 | 4.34 | 1.30 | 2.27 |
| Percentile | | | | | | | | | | | |
| N-ECM | | P < | P = | P < | P < | P < | P = | P = | P < | P = | P = |
| | | 0.0001 | 0.0102 | 0.0001 | 0.0001 | 0.0001 | 0.0077 | 0.0033 | 0.0001 | 0.0429 | 0.0480 |
| D-ECM | | | P < | P = | P = | P = | P = | P = | P = | P = | P = |
| | | | 0.0001 | 0.4530 | 0.8648 | 0.3433 | 0.0836 | 0.0050 | 0.0010 | 0.0365 | 0.7922 |
| D + TGFβi | | | | P < | P < | P < | P = | P = | P < | P = | P = |
| | | | | 0.0001 | 0.0001 | 0.0001 | 0.2062 | 0.1983 | 0.0001 | 0.5006 | 0.1615 |
| D + DMSO | | | | | P = | P = | P = | P = | P = | P = | P = |
| | | | | | 0.3501 | 0.5638 | 0.1128 | 0.0100 | 0.0017 | 0.0360 | 0.7932 |
| TGFβi | | | | | | P = | P = | P = | P = | P = | P = |
| | | | | | | 0.1447 | 0.0733 | 0.0066 | 0.0078 | 0.0213 | 0.6185 |
| DMSO | | | | | | | P = | P = | P = | P = | P = |
| | | | | | | | 0.1840 | 0.0258 | 0.0005 | 0.0606 | 0.8954 |
| αvβ5-i | | | | | | | | P = | P = | P = | P = |
| | | | | | | | | 0.7125 | 0.0025 | 0.6954 | 0.6617 |
| α5β1-i | | | | | | | | | P = | P = | P = |
| | | | | | | | | | 0.0002 | 0.6913 | 0.5332 |
| β5 + α5-i | | | | | | | | | | P = | P = |
| | | | | | | | | | | 0.0001 | 0.0437 |
| α5β1-act | | | | | | | | | | | P = |
| | | | | | | | | | | | 0.4812 |
| IgG | | | | | | | | | | | |
| PDAC | | | | | | | | | | | |
| α-SMA | | | | | | | | | | | |
| stress fiber | N- | D- | D + | D + | | | | | | α5β1- | |
| localization | ECM | ECM | TGFβi | DMSO | TGFβi | DMSO | αvβ5-i | α5β1-i | β5 + α5-i | act | IgG |
| 25% | 0.17 | 0.82 | 0.32 | 0.8469 | 0.71 | 0.72 | 0.14 | 0.51 | 0.83 | 0.41 | 0.92 |
| Percentile | | | | | | | | | | | |
| Median | 0.58 | 1.00 | 0.55 | 1.02 | 0.96 | 1.00 | 0.54 | 0.77 | 1.11 | 0.56 | 1.00 |
| 75% | 1.07 | 1.12 | 0.87 | 1.399 | 1.29 | 1.04 | 0.71 | 1.20 | 1.55 | 0.98 | 1.00 |
| Percentile | | | | | | | | | | | |
| N-ECM | | P < | P = | P = | P = | P = | P = | P = | P = | P = | P = |
| | | 0.0001 | 0.8847 | 0.0036 | 0.0110 | 0.0036 | 0.3443 | 0.0325 | 0.0003 | 0.4429 | 0.0232 |
| D-ECM | | | P = | P = | P = | P = | P < | P = | P = | P < | P = |
| | | | 0.0001 | 0.3578 | 0.9132 | 0.2635 | 0.0001 | 0.0016 | 0.0074 | 0.0001 | 0.4019 |
| D + TGFβi | | | | P < | P = | P < | P = | P = | P < | P = | P = |
| | | | | 0.0001 | 0.0001 | 0.0001 | 0.3820 | 0.0014 | 0.0001 | 0.3265 | 0.0021 |
| D + DMSO | | | | | P = | P = | P < | P = | P = | P < | P = |
| | | | | | 0.5739 | 0.1742 | 0.0001 | 0.0130 | 0.1228 | 0.0001 | 0.3784 |
| TGFβi | | | | | | P = | P < | P = | P = | P = | P = |
| | | | | | | 0.3508 | 0.0001 | 0.0798 | 0.0897 | 0.0003 | 0.6626 |
| DMSO | | | | | | | P < | P = | P = | P < | P = |
| | | | | | | | 0.0001 | 0.0706 | 0.0035 | 0.0001 | 0.5352 |
| αvβ5-i | | | | | | | | P < | P < | P = | P = |
| | | | | | | | | 0.0001 | 0.0001 | 0.0883 | 0.0001 |
| α5β1-i | | | | | | | | | P = | P = | P = |
| | | | | | | | | | 0.0004 | 0.0197 | 0.1311 |
| β5 + α5-i | | | | | | | | | | P < | P = |
| | | | | | | | | | | 0.0001 | 0.0333 |
| α5β1-act | | | | | | | | | | | P = |
| | | | | | | | | | | | 0.0047 |
| IgG | | | | | | | | | | | |
| RCC | | | | | | | | | | | |
| α-SMA | | | | | | | | | | | |
| stress fiber | N- | D- | D + | D + | | | | | | α5β1- | |
| localization | ECM | ECM | TGFβi | DMSO | TGFβi | DMSO | αvβ5-i | α5β1-i | β5 + α5-i | act | IgG |
| 25% | 0.05 | 0.88 | 0.28 | 0.689 | 0.87 | 0.91 | 0.46 | 0.54 | 0.63 | 0.74 | 0.96 |
| Percentile | | | | | | | | | | | |
| Median | 0.29 | 1.00 | 0.72 | 0.89 | 1.03 | 1.05 | 0.67 | 0.89 | 1.00 | 0.93 | 1.06 |
| 75% | 0.67 | 1.10 | 0.97 | 1.03 | 1.17 | 1.16 | 0.96 | 1.06 | 1.33 | 1.54 | 2.19 |
| Percentile | | | | | | | | | | | |
| N-ECM | | P < | P = | P < | P < | P < | P = | P = | P = | P < | P < |
| | | 0.0001 | 0.0221 | 0.0001 | 0.0001 | 0.0001 | 0.0051 | 0.0001 | 0.0002 | 0.0001 | 0.0001 |
| D-ECM | | | P = | P = | P = | P = | P = | P = | P = | P = | P = |
| | | | 0.0058 | 0.1657 | 0.6560 | 0.5042 | 0.0101 | 0.1182 | 0.8107 | 0.7417 | 0.0153 |
| D + TGFβi | | | | P = | P = | P = | P = | P = | P = | P = | P = |
| | | | | 0.0630 | 0.0013 | 0.0006 | 0.6048 | 0.0810 | 0.0135 | 0.0038 | 0.0002 |
| D + DMSO | | | | | P = | P = | P = | P = | P = | P = | P = |
| | | | | | 0.0373 | 0.0091 | 0.0705 | 0.7663 | 0.2730 | 0.2723 | 0.0022 |
| TGFβi | | | | | | P = | P = | P = | P = | P = | P = |
| | | | | | | 0.7996 | 0.1031 | 0.8438 | 0.8330 | 0.8330 | 0.0002 |
| DMSO | | | | | | | P = | P = | P = | P = | P = |
| | | | | | | | 0.0019 | 0.0293 | 0.9733 | 0.5723 | 0.1915 |
| αvβ5-i | | | | | | | | P = | P = | P = | P = |
| | | | | | | | | 0.2704 | 0.0932 | 0.0269 | 0.0001 |
| α5β1-i | | | | | | | | | P = | P = | P = |
| | | | | | | | | | 0.2388 | 0.1797 | 0.0008 |
| β5 + α5-i | | | | | | | | | | P = | P = |
| | | | | | | | | | | 0.8867 | 0.0880 |
| α5β1-act | | | | | | | | | | | P = |
| | | | | | | | | | | | 0.0665 |
| IgG | | | | | | | | | | | |

Significant values P ≤ 0.05;
Marg. Significant values P = 0.10 − 0.05

### Example 3: Integrins αvβ5 and α5β1 Respectively Promote and Impede D-ECM Dependent Myofibroblastic Activation

It was next investigated whether integrins αvβ5 and/or α5β1 were important for D-ECM induced myofibroblastic activation, as these integrin heterodimers are known to participate in myofibroblastic differentiation. Both of these heterodimers were highly abundant on the plasma membranes of naïve and tumor-associated PDAC and RCC fibroblasts (see, FIG. 6F), excluding significant signaling differences related to expression. The alternative hypothesis, that activation of these integrins in naïve fibroblasts following exposure to D-ECM induces myofibroblastic conversion, was also tested.

FIGS. 2A through 2D show that alpha-v-beta-5 (αvβ5) integrin regulates alpha-5-beta-5 (α5β1) integrin activity thus maintaining D-ECM-induced myofibroblastic/desmoplastic phenotype in a TGFβ-independent manner. Naïve/innate PDAC and RCC patient harvested fibroblasts were cultured overnight within normal (N-ECM) vs. desmoplastic (D-ECM or DECMs that were produced in the presence or absence of TGFβ1-receptor inhibition (D+TGFβi) or vehicle control (D+DMSO). Alternatively, naïve cells cultured within D-ECMs were treated with either TGFβ1-receptor inhibitor (TGFβ-i), vehicle (DMSO), functional blocking antibodies anti-αvβ5 integrin (alula, αvβ5-i), anti-α5β1 integrin (mAb16, α5β1-i), combinations of both functional blocking antibodies (β5-i+α5-i), functional stabilizing anti α5β1 integrin (SNAKA51; α5β1-act) or non-immunized isotypic antibodies (IgG). All samples were subjected to indirect immunofluorescent labeling of desmoplastic marker α-SMA and counterstained with fluorescently labeled phalloidin to detect actin stress fibers (F-actin) and determine the co-localization ratio. Monochromatic “heat map” images (see, FIG. 2A) represent α-SMA intensity values. A 255 tones intensity scale bar is shown to the right. A summary of all measured values is graphed in FIG. 2B. Additional monochromatic images indicative of double labeled staining for α-SMA and F-actin are in FIG. 2C while levels of total α-SMA localized at corresponding stress fibers are plotted in FIG. 2D. Untreated D-ECM conditions were included in all experiments summarized in this figure and serve as normalization (1 arbitrary unit; a.u.) controls. Matching values including statistical significances are listed in accompanying Table 1. All scale bars represent 50 μm. TGFβ activity was needed to maintain functional D-ECM production but was dispensable when testing D-ECM induced responses, while the later were obstructed by αvβ5 integrin inhibition or active α5β1 integrin stabilization.

Indeed, inhibition of αvβ5 integrin activity (using Alula functional-blocking antibody) in naïve cells cultured within D-ECM reduced α-SMA expression to the levels found in cells maintained by NECM, particularly in the pancreatic model (see, FIGS. 2A and 2B and Table 1). Alula also reduced α-SMA localization to stress fibers (by 54% and 67% in pancreatic and renal naïve fibroblasts, respectively) (see, FIGS. 2C and 2D and Table 1). In contrast, use of the antibody mAb16 to block α5β1 integrin activity had very limited effects on α-SMA expression or localization, resembling the phenotypes induced by a nonimmunized isotypic antibody used as a negative control (see, FIGS. 2B and 2D and Table 1). Unexpectedly, when Alula and mAb16 were combined, the effects of αvβ5 integrin inhibition (i.e., ALULA alone) were lost, with naïve cells undergoing robust myofibroblastic transition as if plated in D-ECM-induced controls ((IgG) see, FIGS. 2A-2D, and Table 1). These results suggested D-ECM induction of myofibroblastic activation could be inhibited by α5β1 integrin activity, potentially with α5β1 acting downstream of αvβ5.

To test this possibility, whether stabilizing the activity of α5β1 integrin with the antibody SNAKA51 would phenocopy αvβ5 integrin inhibition was tested. Incubation of naïve fibroblasts cultured in DECM with SNAKA51 or isotypic negative control antibodies (Table 1) caused pancreatic cells to assume α-SMA expression and stress fiber localization phenotypes indistinguishable from those induced by plating on N-ECM. A similar, although less pronounced, effect was observed with renal cells (see, FIGS. 2A-2D and Table 1).

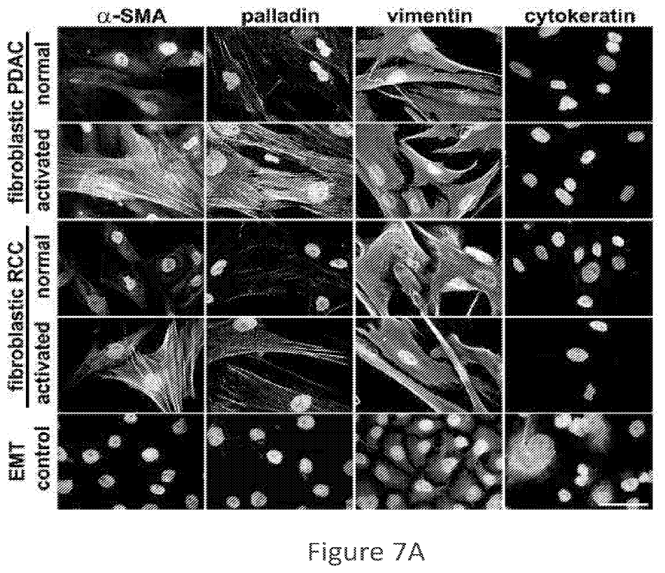

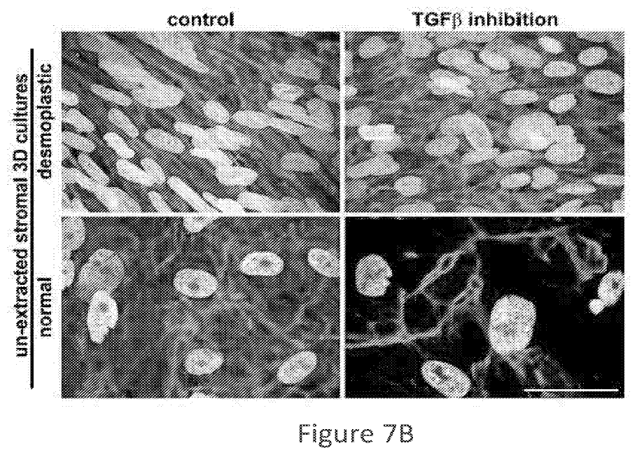

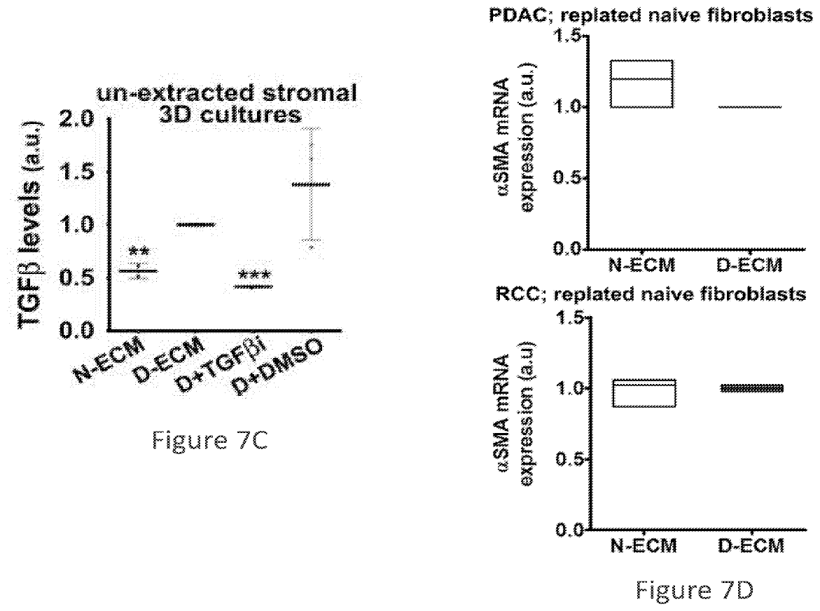

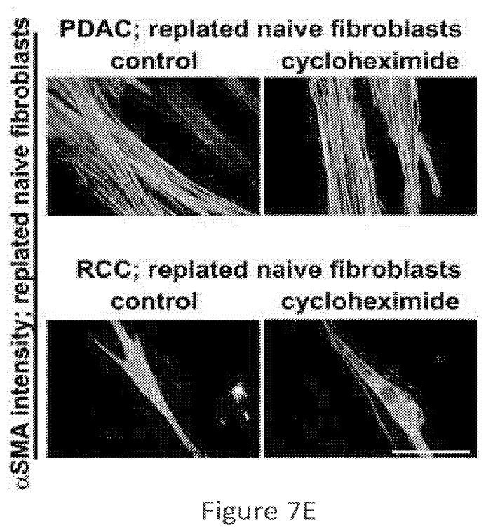

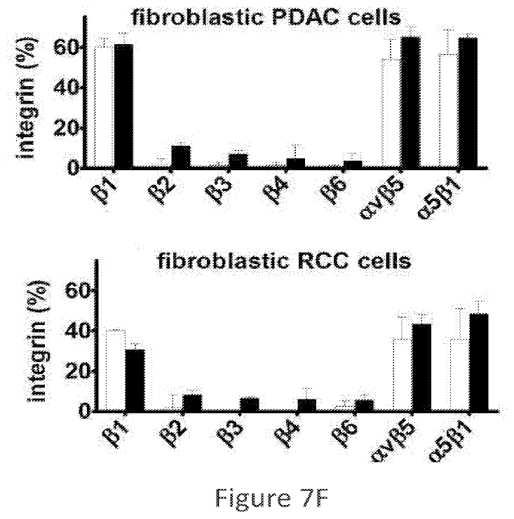

FIGS. 7A through 7F show characterization of human fibroblastic cells isolated from PDAC and RCC surgical samples. FIG. 7A shows representative indirect immunofluorescent assessments of vimentin positive and pan-cytokeratin negative fibroblasts, isolated from PDAC and RCC surgical samples. Isolated cells were probed for desmoplastic markers α-SMA and palladin while Panc1 (a pancreatic cancer cell line) cells were used as epithelial to mesenchyme transduced (EMT) controls known to express both epithelial and mesenchymal markers. Assorted markers are shown in white while counterstained Hoechst identified nuclei are shown in yellow. FIG. 7B shows representative indirect immunofluorescent images of assorted un-extracted (nuclei) ECMs produced in the presence or absence of TGFβ1-receptor blockage. TGFβ inhibition causes disorganization of desmoplastic and ablation of normal ECMs. FIG. 7C shows the TGFβ protein levels from 3D unextracted cultures lysates that were measured by ELISA. Data were normalized to total protein concentration and to D-ECM readouts; results are expressed as arbitrary units (A.U.). FIG. 7D shows α-SMA mRNA levels, assessed via RT-qPCR, of naïve/normal fibroblasts re-plated in corresponding N- vs. D-ECMs were unaltered in both PDAC and RCC models. FIG. 7E shows semi quantitative indirect immunofluorescence showing similar pseudo colors depicting comparable α-SMA protein intensity levels of naïve fibroblasts re-plated in D-ECMs comparing vehicle (control) vs. cycloheximide overnight treatments. FIG. 7F shows integrin-dependent cell adhesion assessment of primary fibroblasts isolated from normal (white bars) vs. matched tumor tissue (desmoplastic; dark bars), from PDAC and RCC surgical samples. All scale bars represent 50 μm.

It was then assessed whether manipulation of integrin activities affected D-ECM production. In contrast to the results seen following incubation with inhibitors of TGFβ, neither αvβ5 integrin inhibition or α5β1 integrin stabilization influenced the properties of D-ECMs (see, FIGS. 7A and 7B), with these matrices being as effective in inducing myofibroblastic activation in naïve fibroblasts as D-ECMs produced in the presence of isotype control antibodies (see, FIGS. 7C and 7D).

In sum, αvβ5 integrin inhibition or stabilization of α5β1 integrin activity blocked the ability of D-ECM to induce myofibroblastic activation of naïve fibroblasts, but failed to significantly influence D-ECM production.

### Example 4: FAK-Independent α5β1 Integrin Activity Blocks D-ECM-Induced Myofibroblastic Activation

Although integrins signal through FAK and SRC kinases, possible FAK-independent roles have been proposed for α5β1 integrin activity. A model of murine D-ECMs (mD-ECMs) was used with fibroblasts wild type or genetically null for FAK or SRC to address the role of these proteins in myofibroblastic activation.

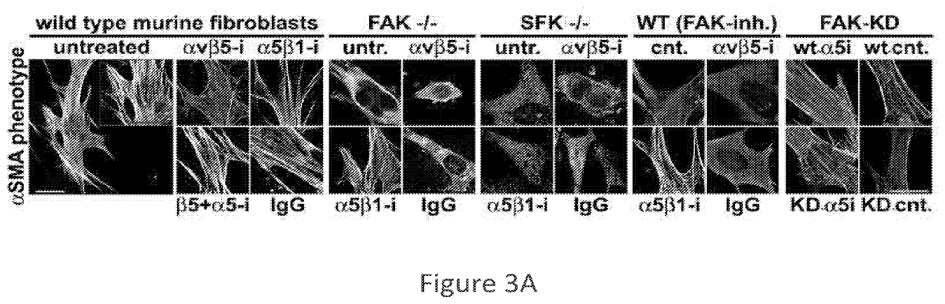

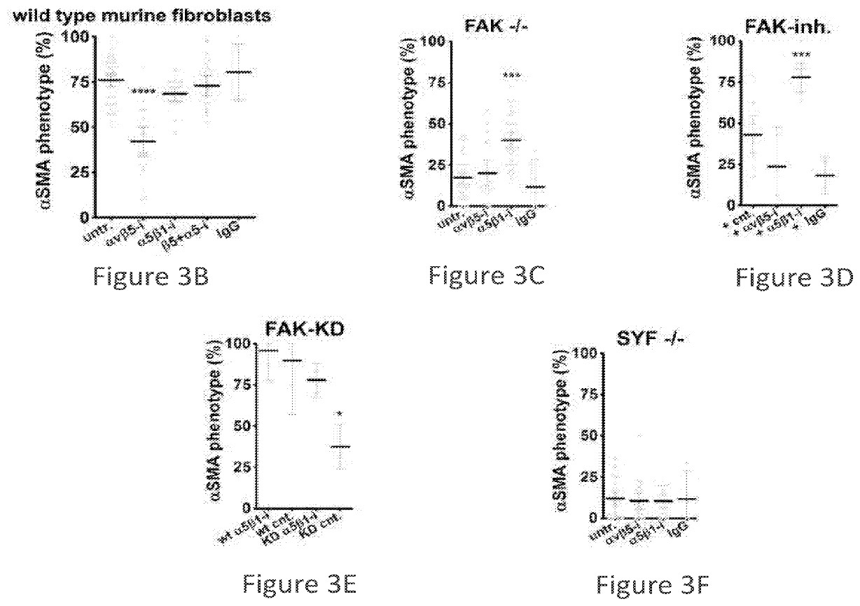

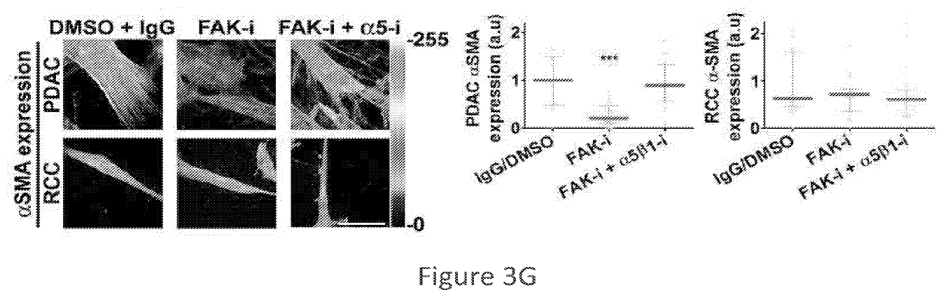

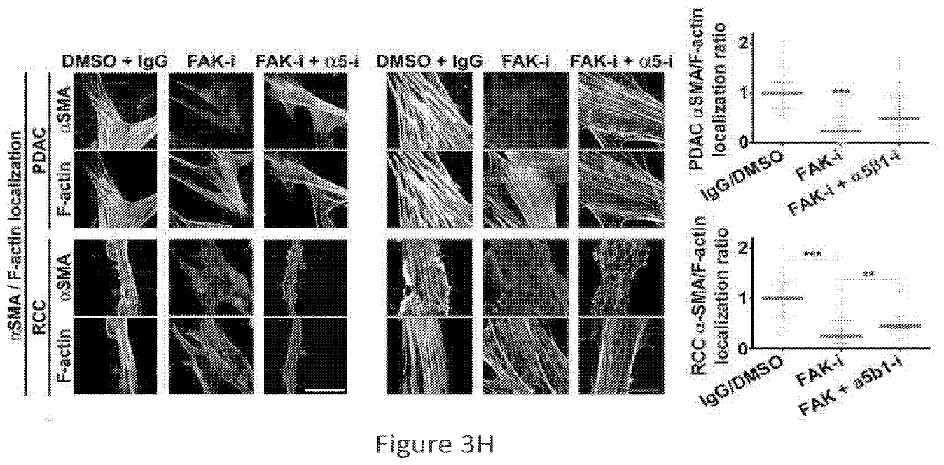

FIGS. 3A through 3H show FAK independent α5β1 integrin activity negatively regulates D-ECM-induced desmoplastic activation. Murine (see, FIGS. 3A-3F) and human (see, FIGS. 3G-3H) extracted D-ECMs were used as substrates and cultured with naïve/innate fibroblasts and challenged to test effects of αvβ5 and α5β1 integrin inhibitions (αvβ5-I and α5β1-I; using alula and/or BMAS (Millipore, Temecula, Calif.) alone or under FAK or SRC family kinase blockage. FAK or SRC family kinase blockage conditions included untreated naïve wild type controls (untr.; see, FIGS. 3A and 3B and FIGS. 3G and 3H), FAK null (FAK−/−; A and C), SRC family kinase null (SYF−/−; see, FIGS. 3A and 3D), naïve wild type cells cultured in the presence of vehicle (cnt.+/−IgG) or small molecule FAK inhibitor (FAK-inh; see, FIGS. 3A and 3E or FIGS. 3G and 3H) and kinase dead FAK mutants (FAK-KD; see, FIGS. 3A and 3F) vs. hTert immortalized littermate controls (wt-cnt.; see, FIGS. 3A and 3F). Murine stress fiber α-SMA phenotypes are shown in 3A while summarized results are depicted in FIGS. 3B-3F graphs. The asterisk shown in the first panel of FIG. 3A represents the zoomed area image depicted as its insert. All bars in FIG. 3A represent 20 microns (μm). Human α-SMA expression and localization levels are shown in FIGS. 3G and 3H, respectively. α5β1 integrin inhibition here was with mAb16 while ALULA was used to inhibit both murine and human αvβ5 integrin forms. Asterisks in FIG. 3H represent areas zoomed in the corresponding panels to the right while graphs in FIG. 3G (right upper and lower panels) summarize all results. Regular scale bars in FIGS. 3G and 3H represent 50 μm while the red scale bar in the zoomed right panels (see, FIG. 3H) corresponds to 15 μm. In all instances tested D-ECM induced phenotype was lost under FAK (or SRC blockage) blockage (or loss), while FAK but not SRC inhibited phenotypes were rescued under α5β1 integrin co-inhibition.

It was first confirmed that myofibroblastic activation by mD-ECMs is similar to that by human D-ECMs in its requirement for active αvβ5—and inhibited α5β1 integrin. FIGS. 3A and 3B show that 80% of murine fibroblasts cultured in mD-ECM underwent myofibroblastic activation, with αvβ5 integrin inhibition significantly reducing this percentage (to—55%, P<0.0001). As in the human models, the phenotype was effectively rescued by the addition of a α5β1 integrin inhibitor (to ˜92% and ˜94% under α5β1 integrin inhibition alone or in combination with αvβ5 integrin inhibition, respectively). mD-ECM induced myofibroblastic activation was significantly reduced by genetic ablation of FAK, observed in only 17% of FAK−/− fibroblasts (see, FIGS. 3A and 3C). Inhibition of α5β1 (40%, P<0.00001), but not αvβ5 integrin (14%, P=0.9148), partly compensated for the FAK null phenotype.

Similar experiments were performed using a small molecule inhibitor of FAK, (PF573,228). Only 32% of cells plated in mD-ECM in the presence of PF573,228 underwent myofibroblast conversion (P<0.0001 compared to vehicle control) (see, FIG. 3D). Concomitant FAK and α5β1 integrin inhibition significantly increased the percentage of cells presenting myofibroblast activation (76%; P=0.0044 compared to FAK inhibition), while an αvβ5 integrin inhibitor failed to rescue this activation (23%; P=0.2681). To further exclude off-target or indirect effects of drug or knockout, we asked whether fibroblasts expressing a dominant negative FAK kinase-dead mutant (FAK-KD) could also be rescued by blocking α5β1 integrin activity. FIG. 3E shows the loss of mD-ECM induced myofibroblastic activation in FAK-KD cells (23%, versus 94% in isogenic hTert immortalized wild type cells), while inhibition of α5β1 integrin rescued myofibroblast activation (55%; p=0.0286).

By comparison, genetic loss of SRC family kinase also inhibited the ability of naïve fibroblasts to undergo mD-ECM-induced myofibroblastic activation. In contrast to FAK null cells, this SRC inhibited phenotype could not be rescued by simultaneous loss of α5β1 integrin activity (FIGS. 3A and 3F). In addition, the SRC family kinase inhibitor, PP2, was used alone or in combination with α5β1 integrin and the D-ECM. Again, this D-ECM-induced phenotype was lost in both human models (not shown).

Results effectively phenocopied α5β1 integrin dependencies using the FAK inhibitor PF573,228 in the PDAC and RCC model systems (see, FIGS. 3G and 3H). FAK inhibition reduced α-SMA expression to 17% or 70% and stress fiber localization levels to 23.5% or 25% in PDAC or RCC models, respectively, and both models showed modest but significant increases in α-SMA stress fiber localization following α5β1 integrin inhibition (PDAC: 49%, P<0.0001 and RCC: 45%, P=0.0051).

Taken together, these results indicated that the negative effect of α5β1 integrin activity on myofibroblast conversion is FAK independent, while SRC activity is indispensable for this process.

### Example 5: D-ECM Influences 3D-Adhesion Structure and αvβ5 Integrin-Regulated Redistribution of Active α5β1 Integrin

Cells grown in N-ECM form 3D-adhesion structures that mediate matrix-dependent homeostasis. 3D-adhesions are elongated adhesion plaques that are highly dependent on α5β1 integrin activity and are characterized by the presence of low constitutive pFAK-Y³⁹⁷.

Whether growth in D-ECM influences the architecture or composition of these structures was evaluated. The median lengths of 3D-adhesions in PDAC and RCC associated fibroblasts were increased in D-ECM by 14% (P<0.0001 n=3633) and 10% (P=0.049, n=698), respectively, compared to N-ECM (see, FIGS. 4A and 4B). Treatment of cells grown in D-ECM with the α5β1 integrin stabilizing agent SNAKA51 reverted adhesion phenotypes similar to those induced by N-ECM.

FIGS. 4A through 4M show molecular alterations in three dimensional matrix adhesions (3D-adhesions) phenotype, as well as αvβ5 integrin-regulated redistribution of active α5β1 integrin to intracellular locations, which constitute trademarks of D-ECM induced responses. Indirect immunofluorescent and spinning disc confocal generated images of 3D-adhesions formed by naïve/innate fibroblastic cells cultured within assorted ECMs in the presence or absence (cnt.) of αvβ5 integrin inhibition (αvβ5-i) or α5β1 integrin stabilization (α5β1 act) are shown in FIG. 4A. Structures in FIG. 4A represent computer-selected internally threshold objects (ITOs) of 3D-adhesion structures and were used for length analysis and for generating the values graphed in FIG. 4B. Double labeled images depicting 3D-adhesion and active α5β1 integrin in naïve fibroblasts cultured under experimental conditions as in FIGS. 4A and 4B are shown in FIG. 4C. Monochromatic/intensity map images in FIG. 4C represent semi quantitative renderings of active α5β1 integrin levels, while a corresponding intensity bar is on the far right. FIG. 4D shows total active α5β1 integrin levels and percentage distributions of total active α5β1 integrin levels at 3D-adhesion locations. FIG. 4E shows total active α5β1 integrin levels, normalized to D-ECM mean intensities, localized at 3D-adhesions. These were calculated using SMIA-CUKIE software (github.com/cukie/SMIA). FIG. 4F shows graphs corresponding to active α5β1 integrin intensity localized away from 3D-adhesions, values were normalized to mean intensity obtained in D-ECM, calculated using SMIA-CUKIE. In FIG. 4G, the size of pie graphs are relative to SMIA-CUKIE output corresponding to total intensity levels, while relative percentage distributions at 3D-structures and away from these are shown. Data were normalized to D-ECM induced distribution percentages (total 100%). Left pie charts represent PDAC values, while right pies correspond to RCC.

FIG. 4H shows representative images depicting 3D-adhesion structures, active α5β1 integrin and pFAK indirect immunofluorescent-labeled locations using permeable experimental conditions in both models. Line histograms to the right depict intensity levels of these there markers (at adhesion structure locations). Scale bar represents 6 μm. FIG. 4I shows graphs depicting calculated levels of active FAK (pFAK) localized at 3D-adhesions structures, calculated using SMIA-CUKIE algorithm. FIG. 4J depicts intracellular vs cell membrane distribution of active α5β1 integrin. Representative indirect immunofluorescent images (PDAC model) of active α5β1 integrin locations relative to 3D-adhesions, under permeable vs. non-permeable conditions to discriminate the mentioned cell surface from intracellular compartments are shown. The third image to the right demonstrates the specificity of active α5β1 integrin detection, as samples were pre-treated with a functional blocking anti α5β1 integrin antibody. Scale bar represents 50 μm. The graph to the right summarizes the observed results. FIG. 4K shows pie graphs of active α5β1 integrin fractions distributed at plasma membrane, at (yellow) or away (light green) of 3D-adhesions, in contrast to intracellular (dark green) locations (which will render the material incorporated in the extracellular vesicles). Diminished levels of intracellular active α5β1 integrin are represented by the “empty” pie wedges (gray). Analysis was performed using SMIA-CUKIE https://github.com/cukie/SMIA. In FIG. 4L, representative PDAC indirect immunofluorescent images corresponding to overnight “chase” incubations with pre-labeled anti active α5β1 integrin antibodies or IgG controls followed by de novo active α5β1 integrin labeling (after cell fixation) relative to 3D-adhesion structures under permeable vs. non-permeable conditions as indicated. FIG. 4M shows PDAC transmitted electron microscopy images of immune gold labeling 3D adhesions (large particles) vs. active α5β1 integrin (small particles) in naïve cells cultured within N- vs. D-ECM in the presence or absence of αvβ5 integrin blockage (αvβ5-i) showing lowering and re-localization of active α5β1 integrin pools. Scale bars: A; monochromatic images and small ITO bars represent 50 μm, while in zoomed ITO images bars represent 10 μm. In FIG. 4H, bar represents 6 μm; in FIG. 4J, bar represents 50 μm; in FIG. 4L, bar represents 10 μm; and in Figure M, bar represents 0.5 μm.

Unexpectedly, semi-quantitative indirect immunofluorescence analysis (using the SMIA-CUKIE software) showed higher levels of activated α5β1 integrin in cells grown in D-ECM versus N-ECM (see, FIGS. 4C and 4D; P<0.00001 in both models). Nonetheless, α5β1 integrin activity levels that were localized at 3D-adhesions remained comparable in N-ECM vs.

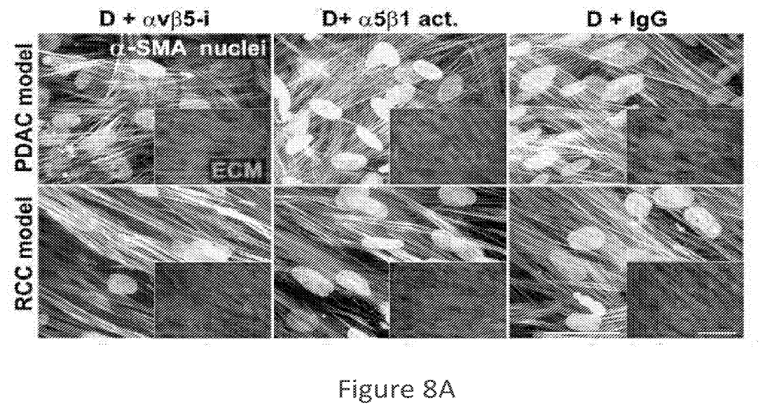

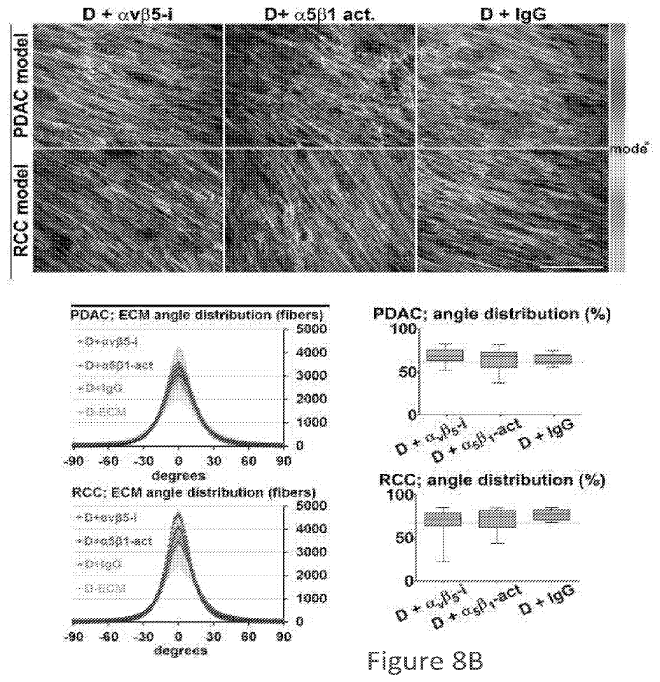

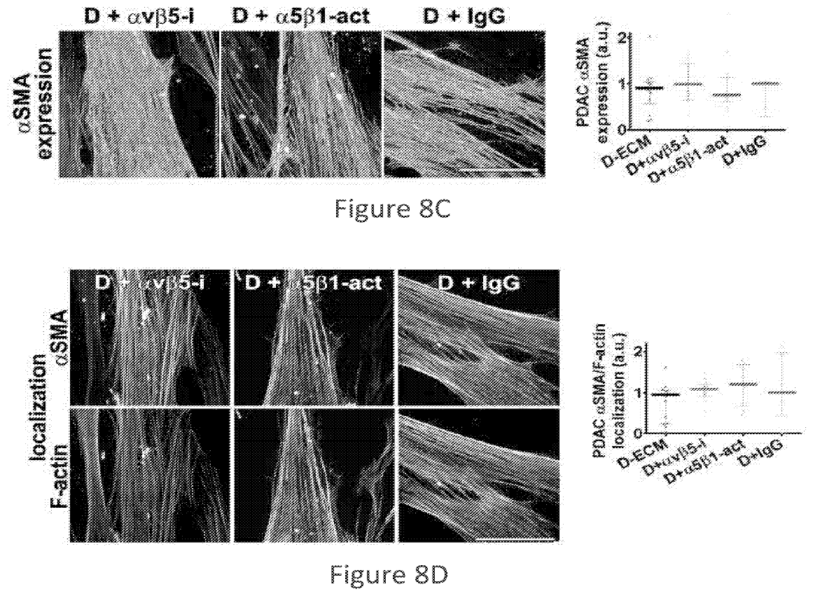

D-ECM (see, FIG. 4E). Therefore, the hypothesis that D-ECM triggers the formation of an “altered” desmoplastic 3D-adhesion and induces a concomitant re-distribution of the observed excess in α5β1 integrin activity away from these structures was tested. For this, a molecular analysis of assorted 3D-adhesions revealed that DECMs prompted increased levels of pFAK-Y³⁹⁷ at 3D-adhesions, compared to levels seen at these locations in response to N-ECMs (2 fold; P<0.0001 in both PDAC and RCC models; see, FIG. 4F). When the same analysis was conducted in the presence of αvβ5 integrin inhibition, pFAK-Y³⁹⁷ levels were restored back to N-ECM-induced values in both models. Images and linear histograms corresponding to these analyses are shown in FIG. 8A.

The same algorithm (SMIA-CUKIE) was used to gauge the intensity levels of α5β1 integrin activity that are localized away from 3D-adhesions (see, FIG. 4G). Results, relative to levels in D-ECMs (a.u.=1), pointed to lower α5β1 integrin activity levels in N-ECMs (PDAC, ˜0.5; RCC 0.2, (P<0.0001). Inhibition of αvβ5 integrin activity restored pools of α5β1 integrin activity localized away from 3D-adhesions to N-ECM induced levels in PDAC (˜0.32, P<0.0001), while the activity distribution was not significantly altered in RCC (0.82, P=0.35). These results are summarized in FIG. 4H.

FIGS. 8A through 8D show αvβ5 integrin inhibition or α5β1 integrin stabilization during D-ECM production failed to alter the resulting ECMs. FIG. 8A shows representative indirect immunofluorescent images of assorted un-extracted D-ECMs produced in the presence of functional blocking anti αvβ5 integrin (alula; D+αvβ5-i), functional stabilizing anti α5β1 integrin (SNAKA51; D+α5β1-act.) or non-immunized isotypic antibodies (D+IgG). Spinning disk confocal monochromatic images, obtained following indirect immunofluorescence, show nuclei (Hoechst), α-SMA and ECM (fibronectin). FIG. 8B shows corresponding ECM fiber angle distributions, using Image-J's “OrientationJ” plug, which were normalized using hue values for a cyan mode angle visualization that is shown in the bar on to the right. Corresponding curves depicting averaged and variations of angle distributions normalized to cero degree modes summarizing results. Plotted data depicting summarized percentages of fibers distributed at 15 degree angles from the mode per experimental condition. FIG. 8C shows pseudo colored images representing α-SMA intensity values; the summary of all measured values is graphed to the right. FIG. 8D shows Monochromatic images indicative of double labeled staining for α-SMA and F-actin are shown while levels of total α-SMA localized at corresponding stress fibers are plotted to the right. None of the treatments seem to have altered D-ECM production (see, FIGS. 8A and 8B) or function (see, FIGS. 8C and 8D). All scale bars represent 50 μm.

Next, to query if the observed D-ECM-regulated increase in α5β1 integrin activity away from 3D adhesions can be explained by its intracellular redistribution, plasma membrane vs. intracellular α5β1 integrin activity levels were measured via a permeable vs. non-permeable immunofluorescent approach. Indeed, results in FIGS. 8B and 8C indicated a ˜5 (PDAC) and ˜40-fold (RCC) reduction in α5β1 integrin activity, prompted by D-ECM, when cells were not permeated. The observed non-permeable D-ECM phenotype suggested intracellular excess in α5β1 integrin activity. As a control, inhibition of α5β1 integrin with mAb16 under permeable conditions effectively reduced activity levels (P<0.00001) indicating that the measured intensities were indeed indicative of α5β1 integrin activity (see, FIG. 8B). Next, intracellular and plasma membrane levels of α5β1 integrin activity were calculated, including sites at or away from altered 3D-adhesions where absence of pFAK-Y³⁹⁷ labeling served as non-permeable control (see, FIG. 8D). Results revealed that the observed increases in α5β1 integrin activity induced in response to D-ECMs effectively established intracellular pools of integrin activity (see, FIG. 4H). Alula was used to block D-ECM-induced responses, and αvβ5 integrin inhibition reduced the intracellular levels of α5β1 integrin activity in both models, albeit more intensively in PDAC, from ˜80% to 0% in PDAC and from ˜90% to ˜60% in RCC.

To further investigate the effect that stabilizing α5β1 integrin activity has on its D-ECM induced localization, SNAKA51 was used to stabilize α5β1 integrin activity, or pre immunized isotypic negative control, and incubated overnight with pancreatic naïve cells in N- and D-ECMs. Permeable vs. non-permeable conditions were then fixed and compared via de-novo direct (pre-labeled) α5β1 integrin activity immunofluorescence detection. Results in FIG. 4I confirmed that, similarly to αvβ5 integrin inhibition, stabilization of α5β1 integrin activity exhausts active α5β1 integrin intracellular pools, while isotypic antibodies failed to alter active integrin localization. Similar results were obtained using Alula to inhibit D-ECM induced αvβ5 integrin activity followed by double labeled immune-gold transmitted electron microscopy FIG. 4J.

Altogether these data suggest that, in response to D-ECM, αvβ5 integrin distributes excess α5β1 integrin activity to intracellular locations (i.e., away from altered 3D-adhesions) concomitant with inducing high pFAK-Y³⁹⁷ levels.

### Example 6: In Vivo Stromal Levels and Distributions of Active α5β1 Integrin, Concomitant with pFAK-Y³⁹⁷ Forecast PDAC and RCC Recurrences

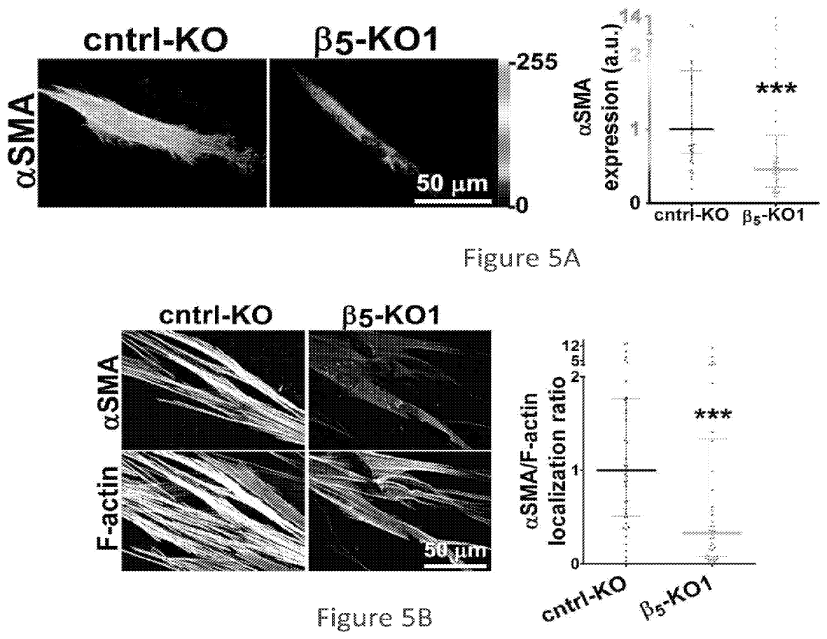

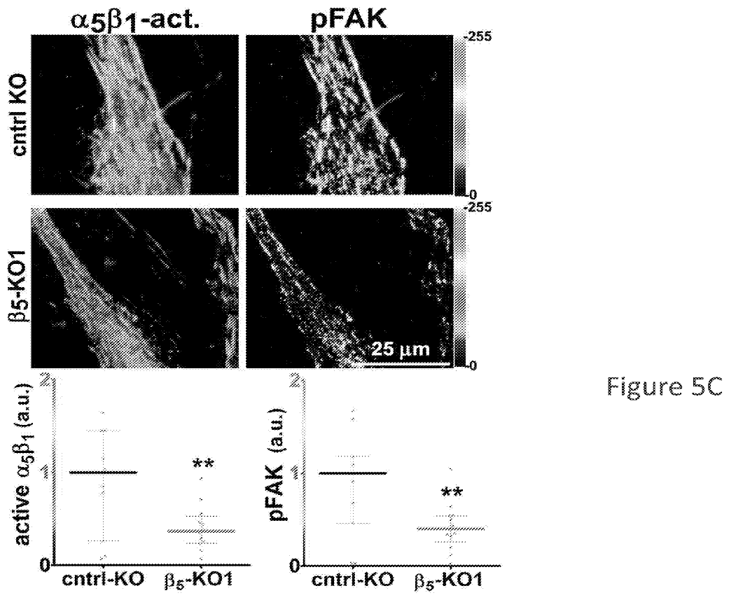

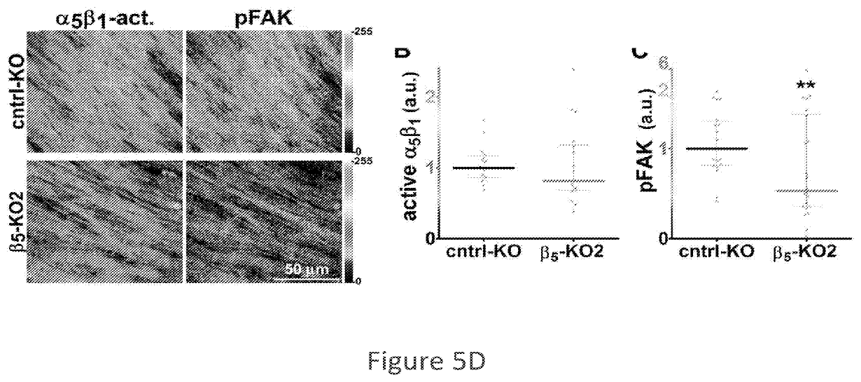

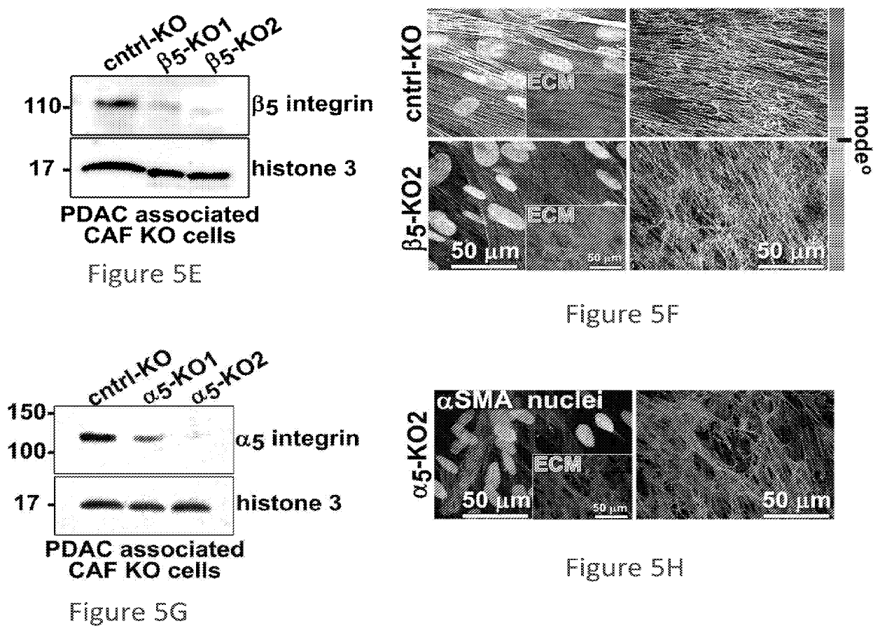

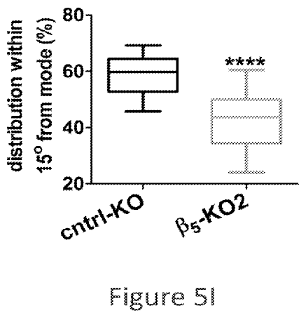

FIGS. 5A-5D show the effect of genetically-nullified 135 integrin gene 035 KO) in fibroblasts reacting to D-ECMs and in D-ECM production (showing only PDAC associated fibroblasts). FIG. 5A shows that naïve 135 KO fibroblasts cultured within D-ECM are impaired to achieve the previously described D-ECM-induced phenotype and display a statistically significant (graphs to the right of the image sets) low α-SMA expression. FIG. 5B shows also that β KO fibroblasts cultured within D-ECM have reduced levels of α-SMA localization at F-actin stress fibers, while FIG. 5C shows decreased levels of both active α5β1 integrin and FAK. FIG. 5D shows β KO CAFs in a native 3D unextracted culture revealing a phenotype with noticeable reduced levels of active FAK and a relocalization of (to the plasma membrane) of active α5β1 integrin. All “heat map” images represent quantified intensities of mentioned markers, and quantified data is expressed in the provided graphs. **Represent statically significant p values (0.01-0.05).

FIGS. 5E-5I show loss of 135 integrin expression in CAFs impairs D-ECM alignment, while expression of α5 integrin is imperative for effective ECM fibrillogenesis. Representative western blot of 135 integrin (see, FIG. 5E) and α5 integrin (see, FIG. 5G) expression in desmoplastic fibroblasts (CAFs), illustrating the result of CRISPR/CAS9-mediated KO of integrins (see, FIG. 5E—β5-KO1+β5-KO2 and FIG. 5G—α5 integrin α5-KO1+α5-KO2) compared to non-targeting gRNA control (cntrl-KO). Histone three was used as a loading control. Representative confocal microscopy images of either control CAF-KO (cntrl-KO) or CAF-β5 integrin-KO2 (β5-KO2) (see, FIG. 5F) or CAF-α5-integrin-KO2 (α5-KO2) (see, FIG. 5H), depicting αSMA (white) and nuclei (yellow). Inserts show the image-matching ECM fibers (fibronectin, magenta). The images on the right show the corresponding ECM fiber angle distributions as obtained using the Image-J's ‘OrientationJ’ plug. Figure SI shows quantification of the distribution of fiber angles that are within 15° of the mode from (FIG. 5F) (****p<0.0001). α5-KO2 CAFs did not produce matrices that were substantial enough for quantification and were therefore omitted from (see, FIG. 50.

Next, it was investigated whether the phenotype induced by D-ECM in naïve fibroblasts, upon re-plating, is also observed in un-extracted 3D TAF cultures during D-ECM production. A simultaneous multi-channel immunofluorescence approach was conducted to concurrently label tumor (absent in vitro), stromal nucleus, and 3D adhesion locations together with levels and localizations of active α5β1 integrin, pFAK-Y³⁹⁷ and pSMAD⅔ (representing TGFβ activation). Results confirmed that un-extracted TAF cultures present high active α5β1 integrin levels that are localized away from pFAK-Y³⁹⁷-positive altered 3D-adhesions concomitant with increased levels of nuclear pSMAD⅔, compared to levels and locations in naïve/normal pancreatic and renal fibroblastic cultures (see, FIG. 8E; high magnification confocal generated image examples are in FIG. 5A).

It was evaluated if the in vitro phenotypes described above effectively simulated in vivo intratumoral stroma pathophysiology. For this, SMIA was conducted using formalin fixed paraffin embedded (FFPE) samples matching the tissues used to generate the in vitro models presented above. In vivo SMIA-CUKIE calculated outputs indicated ˜3 and ˜4 fold (P=0.0013 and P<0.0001) increase in total α5β1 integrin activity, ˜2 and ˜5 (P=0.0311 and P<0.0001) in pFAK-Y³⁹⁷ and ˜1.4 and ˜3 (NS and P=0.0031) fold increases in pSMAD⅔ corresponding to PDAC and RCC intratumoral stroma compared to normal pancreatic and renal parenchyma, respectively (see, FIGS. 5B and 5C). When the same comparisons were measured for α5β1 integrin activity specifically localized away from 3D-adhesions, representative of activated desmoplasia, results indicated ˜2 (PDAC; P=0.0371) and ˜5 (RCC; P<0.0001) fold increases (see, FIGS. 5B and 5D). Together, the observed intratumoral stroma biomarker locations and levels matched concomitant distribution phenotypes observed in vitro, thus confirming the models' in vivo mimicry capabilities.

It was assessed if levels and localization of these stromal biomarkers are clinically prognostic for PDAC and RCC, and whether these tests discriminated activated vs. innocuous intratumoral desmoplastic phenotypes. Using PDAC and RCC tissue microarrays (TMAs; Table 2), high throughput MIA image acquisitions and quantitation of all TMA samples were conducted. Custom-programmed software was used, and the outputs were integrated with corresponding clinical data indicative of overall survival (OS) and recurrence free survival (RFS) for PDAC or Disease Specific Survival (DSS) for RCC (see, FIGS. 5E-54

TABLE 2
Human PDAC and RCC cohorts included in TMAs.

| PDAC | | | RCC | | |
|---|---|---|---|---|---|
| Samples | Number | | Samples | Number | |
| Normal | 17 | | Normal | 19 | |
| Tumor | 67 | | Tumor | 125 | |
| Gender | % | | Gender | % | |
| Female | 56 | | Female | 30.5 | |
| Male | 44 | | Male | 69.5 | |
| Average age | | | Average age | | |
| 67.06 (46-91) | years (range) | | 60.79 (23-82) | years (range) | |
| TNM stage | | | TNM stage | | |
| N | cases (%) | | N | cases (%) | |
| 0 | 27 | (40.30) | 0 | 97 | (77.60) |
| 1 | 38 | (56.72) | 1 | 2 | (1.60) |
| 2 | 0 | (0) | 2 | 10 | (8.00) |
| 3 | 0 | (0) | 3 | 0 | (0) |
| N/A | 2 | (2.99) | N/A | 16 | (12.80) |
| T | cases (%) | | T | cases (%) | |
| 0 | 0 | (0) | 0 | 0 | (0) |
| 1 | 8 | (11.94) | 1 | 34 | (27.20) |
| 2 | 20 | (29.85) | 2 | 33 | (26.40) |
| 3 | 33 | (49.25) | 3 | 46 | (36.80) |
| 4 | 4 | (5.97) | 4 | 0 | (0) |
| N/A | 2 | (2.99) | N/A | 12 | (9.60) |
| M | cases (%) | | M | cases (%) | |
| 0 | 0 | (0) | 0 | 82 | (65.60) |
| 1 | 1 | (1.49) | 1 | 33 | (26.40) |
| N/A | 2 | (2.99) | N/A | 10 | (8.00) |
| Overall stage | cases (%) | | Overall stage | cases (%) | |
| I | 15 | (22.39) | I | 34 | (27.20) |
| II | 27 | (40.30) | II | 27 | (21.60) |
| III | 18 | (26.87) | III | 32 | (25.60) |
| IV | 4 | (5.97) | IV | 22 | (17.60) |
| N/A | 3 | (4.47) | N/A | 22 | (14.76) |

N/A: not available

For PDAC, it was first confirmed that the cohort was representative of known patient outcome distributions, applying univariate analyses to detect associations between pathological cancer stages and OS. Results rendered a Hazard Ratio (HR) of 1.6 (P=0.0021; 95% CI 1.2-2.2). Univariate analyses of PDAC's pathological T and N presented shorter OS times as these increased (T: HR=1.4, P=0.04, 95% CI 1.01-2.0 and N: HR=2.9, P=0.0007, 95% CI 1.6-5.4). Univariate (Uni) and multivariable (MVA) analyses suggested that shorter PDAC RFS times correlate with advanced pathological stages (Uni: HR=2.5, P=2.09E-05 and 95% CI 1.6-3.9; MVA HR=2.6, P=8.18E-05 and 95% CI 1.6-4.3). While none of the tested SMIA-acquired stromal marker levels correlated with changes in OS, analyses conducted using classification and regression trees (CART) showed increased RFS if PDAC surgical samples (which were selected to assure inclusion of mostly tumoral areas) presented with high intratumoral stromal α5β1 integrin activity localized at 3D-adhesions. Observations were similar when SMIA-generated stromal median levels, percentage area coverages or integrated intensities were evaluated; rendering RFS benefits of about 11 months (P=0.076, P=0.064 and P=0.079, respectively) for this phenotype. The resultant PDAC RFS outcome curves representative of percentage stromal coverage areas, positive for active α5β1 integrin localized at 3D-adhesions, are shown in FIG. 5E. Outcomes suggested that specific stromal localization of α5β1 integrin activity at 3D-adhesions was indicative of a protective desmoplastic phenotype (reminiscent of normal stroma) in PDAC patients undergoing curative intended surgery.

For RCC, by applying both univariate and multivariable analyses, searching for associations between pathological cancer stages and OS rendered HRs of 2.4 (P=1.11E-10; 95% CI 1.9-3.2) and 3.2 (P=0.001; 95% CI 1.6-6.2), respectively. Univariate analyses of RCC's pathological T, N and M presented shorter OS times as these increased (T: HR=2.0, P=4.16E-05, 95% CI 1.4-2.8, N: HR=2.1, P=0.0003, 95% CI 1.4-3.1 and M: HR=5.4, P=3.79E-10, 95% CI 3.2-9.1). Similarly, univariate and multivariable analyses suggested that shorter RCC DSS times correlate with advanced pathological stages (Uni: HR=3.1, P=7.94E-11 and 95% CI 2.2-4.3; MVA HR=3.4, P=0.0017 and 95% CI 1.6-7.3). CART generated Kaplan-Meier curves assessing OS and DSS indicated that RCC patients presenting stromal phenotypes reminiscent to activated desmoplasia, such as high stromal levels of pFAK and active α5β1 integrin, respectively, localized at or away from 3D-adhesions, endured significantly shorter times. Specifically, OS of patients presenting with median values of intratumoral stroma pFAK (at 3D-adhesion positive pixels) of more than 130, out of a possible maximum of 255, was just short of 3 years (˜35 months) following surgery, while median survival of patients presenting with lower stromal pFAK intensities was ˜14 years (˜145 months) (see, FIG. 5F).

Similarly, when looking at DSS, the exact same active stromal phenotype rendered RCC patients with only ˜3 years (35 months) following surgery, while patients with low stromal pFAK levels at time of surgery succumbed to RCC significantly later (median DSS of over 15 years; see, FIG. 5G). Concomitantly, the OS and DSS of patients that presented with high active α5β1 integrin localized away from 3D-adhesions was just short of 4 and 5 years, while patients with low active integrin intensities at these locations presented median survival of ˜15.5 years and longer than 16.6 years (the maximum follow up time), respectively (see, FIGS. 5H and 5I). In summary, RCC patients presenting high active desmoplastic levels, based on both high constitutive stromal pFAK at 3D adhesions and α5β1 integrin activity away from 3D-adhesions, tended to present poorest outcomes following surgery.

Together, these results suggest clear predispositions for newly identified active desmoplastic phenotypes to predict patient outcomes; inactive phenotype foresees longer time periods until PDAC recurrence, while active phenotypes predict shorter survival times in RCC.

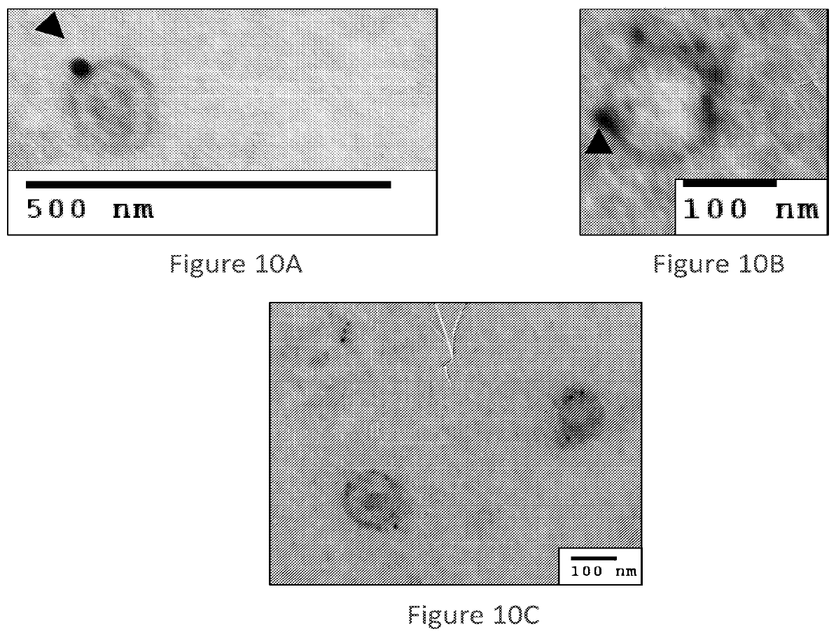

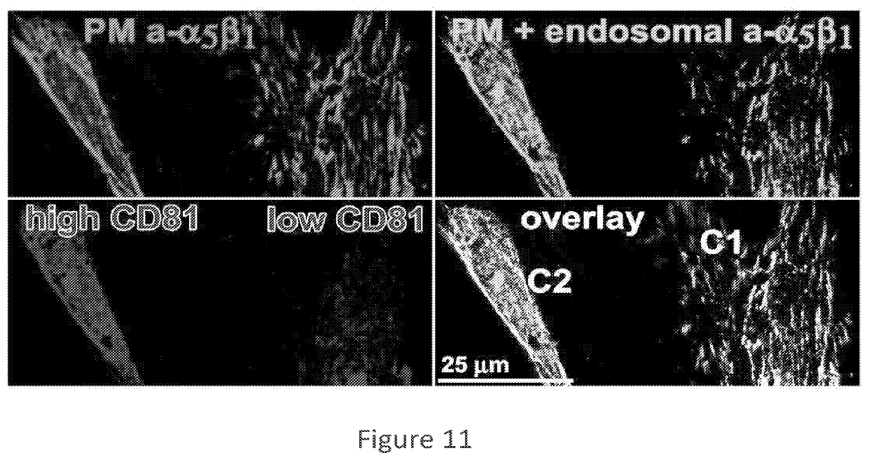

FIGS. 10A-10C depict images of isolated cell-secreted microvesicles/exosomes containing active α5β1 integrin at the vesicle membrane. FIG. 10A shows exosomal vesicles collected from conditioned media of 3D unextracted CAFs in culture. Electron microscopy scanning revealed the characteristic morphology (cup shape) of exosomes at the known/distinct size of these vesicles (60-140 nm). FIGS. 10A and 10B (arrowheads) show positive detection of active α5β1 integrin using SNAKA51 antibody linked to large (Au10) electrodense gold particles. Known exosomal markers were used to further characterized these vesicles and confirmed presence of CD-81 (small arrows pointing to Au6 particles). FIG. 10C shows CD-81/CD-63 positive exosomal vesicles purified from murine serum. Scale bars are provided.

FIG. 11 confirms not only that active α5β1 integrin is intracellularly localized at multi-vesicular bodies of activated CAFs (C2), marked by CD81, but that loss of CD81 prompts the relocaltion of the integrin at the plasma membrane (C1; representing normalized fibroblasts akin to αvβ5 inhibition). CD81 siRNA-depleted C2-CAFs cultured in D-ECM. PM localized a-α5β1 was visualized with SNAKA51 (red) at 4° C., followed by fix and permeabilized incubation with additional SNAKA51 (green) and anti-CD81 (blue). Low CD81 levels (cell on right) correlate with C1 (normalized) phenotype; increased plasma membrane (PM) and reduced endocytic activated α5β1.

The mesenchymal stroma of both PDAC and RCC have long been considered a particularly important component of these neoplasias. A significant portion of PDAC's tumor mass is composed by a cellular fibrous desmoplastic reaction, while activated/myofibroblastic stromal cells are normally intercalated within RCC's tumor components. A dual, permissive and protective, role has been proposed for desmoplastic stroma, while the tumor suppressive role of normal/innate mesenchymal stroma has long been appreciated in epithelial cancers. Relatedly, fibrotic chronic wound healing is known as a predisposing neoplastic condition.

In the foregoing Examples, it is shown that the use of primary fibroblast-derived ECM 3D models bestows the possibility to study both ECM production/remodeling as well as ECM-induced cell responses. Hence, the study first focused on effects of TGFβ, as it is known to regulate myofibroblastic differentiation, and then on integrins αvβ5 and α5β1, which play a role in this process. Whether blocking signals from these pathways could serve as potential desmoplasia reversion targets and the uncovered desmoplastic phenotypes were tested and, based on stromal localization of active α5β1 integrin, serve as new clinical diagnostic and/or prognostic biomarkers.

It was observed that TGFβ is necessary for N-ECM assembly, while it is essential solely for the phenotypic remodeling of D-ECM and not its production. These results are in agreement with published work showing that ECM changes are necessary to achieve TGFβ-dependent myofibroblastic activation associated with increased fibrous-like collagen I production. To this end, levels of D-ECM parallel alignments, reminiscent to observed in vitro D-ECM production phenotypes, correlate with poor patient survival and cancer relapses. Nonetheless, these data suggest that while TGFβ-targeted therapeutic interventions could very well revert D-ECMs back to their naturally tumor-suppressive phenotype, the same approach could result in N-ECM damage.

Conversely, the model renders it possible to assess D-ECM-induced desmoplastic responses where TGFβ activity is dispensable. The data show that D-ECM-dependent myofibroblastic activation, a SRC family kinase dependent process, is maintained as opposed to being induced by a crosstalk between integrins αvβ5 and α5β1. It was found that D-ECM induces increases in active pools (but not expression) of α5β1 integrin which, in turn, can revert D-ECM desmoplastic activation in a FAK independent manner. The results suggest that D-ECM regulated αvβ5 integrin causes a redistribution of α5β1 integrin activity to endogenous pools, thus promoting maintenance of SRC dependent D-ECM induced desmoplastic activation. The results suggest that the α5β1 integrin impediment of D-ECM-induced desmoplastic activation is FAK independent, while de novo α-SMA protein synthesis is dispensable during maintenance of the active phenotype. The in vitro work also rendered phenotypic stromal features whereby cellular stromal localization of α5β1 integrin activity in relation to 3D-adhesions can be probed in vivo.

The results in these Examples begin to consolidate pending uncertainties in the desmoplasia field; similar to published data, percentage coverages of active stroma or stroma generally correlated with improved patient survival. Nevertheless, intensities and localization of active α5β1 integrin concomitant with activated FAK correlated with increasingly aggressive and unfavorable outcomes. The results suggested that while the specific stromal location of active α5β1 integrin activity pools is important in PDACs, increases in stromal activity alone suffice for predicting RCC recurrences. Hence, location and intensities of stromal α5β1 integrin activity are believed to represent novel molecular categories of elicited desmoplastic patient-detrimental stroma, while coverage of stroma fundamentally constitutes a patient protective trait.

In sum, the results suggest that αvβ5 integrin prevents FAK-independent α5β1 integrin activity from impairing D-ECM induced myofibroblastic activation. The results also suggest that levels and localization of stromal α5β1 integrin activity concomitant with FAK could assist in stratifying individuals that could benefit from αvβ5 integrin tampering therapeutics.

The present disclosure is not limited to the embodiments described and exemplified above, but is capable of variation and modification within the scope of the appended claims.

1. A method of inducing desmoplastic stroma to express a normal phenotype comprising:
detecting increased levels of active alpha-5-beta-1 integrin localized intracellularly away from three dimensional matrix adhesions in desmoplastic stroma isolated from a pancreas or kidney of a human subject;
detecting increased focal adhesion kinase activity in the stroma; and
treating the human subject with a therapeutic regimen that inhibits at least one of: fibroblast activation, the biologic activity of transforming growth factor (TGF) beta, and the biologic activity of alpha-v-beta-integrin in pancreatic stroma, or that blocks chronic fibrosis or inflammation, thereby inducing the desmoplastic stroma to express a normal phenotype.

2. The method according to claim 1, wherein the step of detecting increased levels of active alpha-5-beta-1 integrin comprises contacting the isolated desmoplastic stroma with an antibody that specifically binds to an active conformation of alpha-5-beta-1 integrin.

3. The method according to claim 2, wherein the antibody that specifically binds to an active conformation of alpha-5-beta-1 integrin is SNAKA51.

4. The method according to claim 1, further comprising isolating desmoplastic stroma from the pancreas or kidney of the subject by taking a biopsy of the pancreas or kidney.

5. The method according to claim 4, wherein the biopsy is a core needle biopsy or a surgical biopsy.

6. The method according to claim 1, wherein the pancreas or kidney is cancerous.

7. A method of inducing desmoplastic stroma to express a normal phenotype comprising:
detecting active alpha-5-beta-1 integrin in a micro vesicle or exosome of blood or ascites fluid isolated from a human subject; and
treating the human subject with a therapeutic regimen that inhibits at least one of: fibroblast activation, the biologic activity of transforming growth factor (TGF) beta, and the biologic activity of alpha-v-beta-integrin in desmoplastic stroma, or that blocks chronic fibrosis or inflammation, thereby inducing the desmoplastic stroma to express a normal phenotype.

8. The method according to claim 7, further comprising solubilizing the micro vesicle or exosome.

9. The method according to claim 7, wherein the step of detecting active alpha-5-beta-1 integrin comprises contacting the internal contents of the micro vesicle or the exosome with an antibody that specifically binds to an active conformation of alpha-5-beta-1 integrin.

10. The method according to claim 9, wherein the antibody that specifically binds to an active conformation of alpha-5-beta-1 integrin is SNAKA51.

11. The method according to claim 7, wherein the desmoplastic stroma is desmoplastic stroma from the pancreas or kidney.

12. The method according to claim 7, wherein the human subject has pancreatic cancer or kidney cancer.

13. A method of treating a human subject having pancreatic cancer or kidney cancer comprising:
detecting increased levels of active alpha-5-beta-1 integrin localized away from three dimensional matrix adhesions in desmoplastic stroma isolated from a cancerous pancreas or cancerous kidney of the human subject;
detecting increased focal adhesion kinase activity in the stroma;
administering to the human subject an amount of interferon gamma effective to prime T lymphocytes;
isolating desmoplastic stroma from the pancreas or kidney after a period of time following the administering of the interferon gamma;
detecting decreased levels of active alpha-5-beta-1 integrin localized away from three dimensional matrix adhesions in the isolated desmoplastic stroma;
detecting decreased focal adhesion kinase activity in the stroma; and
administering to the human subject an effective amount of an antibody that specifically binds to PD-1 or to PD-L1, thereby treating the pancreatic cancer or kidney cancer.

14. The method according to claim 13, further comprising treating the human subject with a therapeutic regimen that inhibits at least one of: fibroblast activation, the biologic activity of transforming growth factor (TGF) beta, and the biologic activity of alpha-v-beta-integrin in pancreatic stroma.

15. The method according to claim 13, wherein the step of detecting increased or decreased levels of active alpha-5-beta-1 integrin comprises contacting the isolated desmoplastic stroma with an antibody that specifically binds to an active conformation of alpha-5-beta-1 integrin.

16. The method according to claim 15, wherein the antibody is SNAKA51.

17. The method according to claim 13, wherein isolating desmoplastic stroma from the pancreas or kidney of the human subject comprises taking a biopsy of the pancreas or kidney.

18. The method according to claim 17, wherein the biopsy is a core needle biopsy or a surgical biopsy.

19. A method of treating a human subject having pancreatic cancer or kidney cancer comprising:
detecting increased levels active alpha-5-beta-1 integrin in the micro vesicle or exosome from the blood or ascites fluid isolated from the human subject;
administering to the human subject an amount of interferon gamma effective to prime T lymphocytes;
isolating a second micro vesicle or exosome from the blood or ascites fluid of the human subject after a period of time following the administering of the interferon gamma;
detecting decreased levels of active alpha-5-beta-1 integrin in the micro vesicle or exosome; and
administering to the human subject an effective amount of an antibody that specifically binds to PD-1 or to PD-L1, thereby treating the pancreatic cancer or kidney cancer.

20. The method according to claim 19, further comprising solubilizing the micro vesicle or exosome.

21. The method according to claim 19, wherein the step of detecting increased or decreased levels active alpha-5-beta-1 integrin comprises contacting the internal contents of the micro vesicle or the exosome with an antibody that specifically binds to an active conformation of alpha-5-beta-1 integrin.

22. The method according to claim 21, wherein the antibody that specifically binds to an active conformation of alpha-5-beta-1 integrin is SNAKA51.


SEQUENCE LISTING (10 sequences)

SEQ ID NO: 1 - DNA, 20 bp, Artificial Sequence
  gRNA1 for beta 5 integrin
accgagaggtgatggaccgt

SEQ ID NO: 2 - DNA, 20 bp, Artificial Sequence
  gRNA2 for beta 5 integrin
caccgagaggtgatggaccg

SEQ ID NO: 3 - DNA, 20 bp, Artificial Sequence
  gRNA for eGFP
catgtgatcgcgcttctcgt

SEQ ID NO: 4 - DNA, 25 bp, Artificial Sequence
  Integrin beta 5 gRNA 1.1
caccgaccgagaggtgatggaccgt

SEQ ID NO: 5 - DNA, 25 bp, Artificial Sequence
  Integrin beta 5 gRNA 1.2
aaacacggtccatcacctctcggtc

SEQ ID NO: 6 - DNA, 25 bp, Artificial Sequence
  Integrin beta 5 gRNA 2.1
caccgcaccgagaggtgatggaccg

SEQ ID NO: 7 - DNA, 25 bp, Artificial Sequence
  Integrin beta 5 gRNA 2.2
aaaccggtccatcacctctcggtgc

SEQ ID NO: 8 - DNA, 25 bp, Artificial Sequence
  eGFP gRNA 1.1
caccgcatgtgatcgcgcttctcgt

SEQ ID NO: 9 - DNA, 25 bp, Artificial Sequence
  eGFP gRNA 1.2
aaacacgagaagcgcgatcacatgc

SEQ ID NO: 10 - DNA, 21 bp, Artificial Sequence
  U6 promoter forward primer
gagggcctatttcccatgatt